(19) United States
(12) Patent Application Publication
(10) Pub. No.: US 2025/0362218 A1
(43) Pub. Date: Nov. 27, 2025

(54) PARTICLE COUNTING AND BIOMASS MEASUREMENTS OF AGGREGATED CELL COMPOSITIONS

(71) Applicant: FUJIFILM Cellular Dynamics, Inc., Madison, WI (US)

(72) Inventors: Randall LEARISH, Madison, WI (US); Caitlin HIELSBERG, Madison, WI (US); Madison ROGERS, Madison, WI (US); Matt STERNFELD, Madison, WI (US)

(73) Assignee: FUJIFILM Cellular Dynamics, Inc., Madison, WI (US)

(21) Appl. No.: 19/214,864

(22) Filed: May 21, 2025

Related U.S. Application Data
(60) Provisional application No. 63/650,277, filed on May 21, 2024.

(51) Int. Cl. G01N 15/12; C12Q 1/37; G01N 15/10

(52) U.S. Cl. CPC G01N 15/12; C12Q 1/37; G01N 2015/1006; G01N 2015/1024; G01N 2015/1029; G01N 2333/922; G01N 2333/976

(57) ABSTRACT

Provided herein are methods for using particle counting to determine live cell concentration, total cell concentration, cell viability and biomass of cell aggregate compositions, such as for accurate cell dosing for clinical applications.

### PRIORITY CLAIM

This application claims benefit of priority to U.S. Provisional Application Ser. No. 63/650,277, filed May 21, 2024, the entire contents of which are hereby incorporated by reference.

### BACKGROUND

### 1. Field

The present disclosure relates generally to the field of stem cell biology. More particularly, it concerns methods of determining cell viability of a composition comprising cell aggregates.

### 2. Description of Related Art

The use of automated cell counting instruments, such as the ViCell XR Cell Counter or the CELLACA™™ MX high-throughput cell counted, distinguish live and dead cells by use of labeling dyes. Following a labeling step, digital images are captured and analyzed by algorithms that identify and classify objects based on size, labeling intensity, and roundness, etc., and provide a quantitative output for the concentration of live and dead cells. For cell therapy applications, in consideration of the need to formulate a clinical “dose” of cells of known concentration, it is important to validate the use of a counting instrument and the associated software. The ViCell and CELLACA™ instruments work well when cells are individualized (or dissociated). However, their algorithms have limited value when the test samples are in the form of cell clusters or 3D aggregates. Thus, there is an unmet need for a method for determining cell viability of compositions comprising cell aggregates.

### SUMMARY

In a first embodiment, the present disclosure provides a method of obtaining a live cell concentration of a cell aggregate composition comprising:
(a) obtaining a fraction of the cell aggregate composition;
(b) contacting the fraction of the cell aggregate composition with a dissociation enzyme to obtain a dissociated sample; and
(c) quantifying the number of live cells in the dissociated sample by counting the number of particles based on size to obtain a live cell concentration.

In some aspects, the cell aggregate composition comprises cells derived from induced pluripotent stem cells (iPSCs). In certain aspects, the cells derived from iPSCs are photoreceptor precursor cells (PRPs), photoreceptor cells (PRs), or retinal epithelial cells (RPEs).

In certain aspects, the fraction is 1%-5% (e.g., about 1%, 2%, 3%, 4%, or 5%) of the cell aggregate composition. In particular aspects, the fraction is 1% of the cell aggregate composition. In some aspects, the dissociation enzyme is TRYPLE™™, ACCUTASE®, trypsin, dispase, or papain. The dissociation enzyme may be used in conjunction with ethylenediaminetetraacetic acid (EDTA). In additional aspects, the method further comprises treating the dissociated sample with a nuclease, such as a DNAse and/or RNAse, prior to step (c). The nuclease may be benzonase. In certain aspects, the method further comprises triturating the dissociated sample treated with a nuclease.

In some aspects, quantifying the number of live cells comprises using a particle counting instrument configured for detection of single cells, such as the Multisizer 4e coulter counter. In particular aspects, the instrument configured for detection of single cells comprises counting particles with a diameter greater than 6.3 μm (e.g., greater than 6.4, 6.5, 6.7, 6.7, 6.8, 6.9, 7.0, 7.1, 7.2, 7.3, 7.4, 7.5, 8, 8.5, 9, 9.5, or 10 μm).

In certain aspects, the method further comprises adjusting the live cell concentration to the total cell aggregate composition to obtain a target concentration for dose formulation. In some aspects, the cell aggregate composition comprises individual cells, cell clusters, and cell aggregates. In certain aspects, the cell aggregate composition comprises cell clusters and/or cell aggregates. In particular aspects, the method does not comprise using a labeling dye, such as Trypan Blue, acridine orange (AO)/propidium iodide (PI), Hoechst, 4′,6-diamidino-2-phenylindole (DAPI), Phycoerythrin (PE), Allophycocyanin (APC), Fluorescein isothiocyanate (FITC), Carboxyfluorescein diacetate (CFDA), Calcein AM, or 7-aminoactinomycin D (7AAD).

A further embodiment provides a method of obtaining a total cell concentration of a cell aggregate composition comprising:
(a) obtaining a fraction of the cell aggregate composition;
(b) contacting said fraction of the cell aggregate composition with a cell lysis solution to obtain a lysed sample; and
(c) quantifying the number of nuclei in the lysed sample by counting the number of particles based on size to obtain a total cell concentration.

In some aspects, the fraction is 1%-5% (e.g., 1%, 2%, 3%, 4%, or 5%) of the cell aggregate composition. In particular aspects, the fraction is 1% of the cell aggregate composition. In some aspects, the cell lysis solution is Solution 10 (i.e., an acidic aqueous solution of surfactant and organic acid at a pH value in the range of 2-3). In some aspects, quantifying the number of nuclei comprises using a particle counting instrument configured for detection of nuclei. In certain aspects, the instrument configured for detection of nuclei comprises counting particles with a diameter greater than 2.5 μm (e.g., greater than 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4., 4.5, 4.6, 4.7, 4.8, 4.9, or 5.0 μm, such as between 2.8 to 4.8 μm).

In certain aspects, the cell aggregate composition comprises individual cells, cell clusters, and cell aggregates. In some aspects, the cell aggregate composition comprises cell clusters and/or cell aggregates. In certain aspects, the method does not comprise using a labeling dye, such as labeling dye is Trypan Blue, acridine orange (AO)/propidium iodide (PI), Hoechst, 4′,6-diamidino-2-phenylindole (DAPI), Phycoerythrin (PE), Allophycocyanin (APC), Fluorescein isothiocyanate (FITC), Carboxyfluorescein diacetate (CFDA), Calcein AM, or 7-aminoactinomycin D (7AAD).

A further embodiment provides a method of determining the cell viability of a cell aggregate composition comprising:
(a) obtaining a live cell concentration by the method of the present embodiments or aspects thereof; and
(b) obtaining a total cell concentration by the method of the present embodiments or aspects thereof, wherein cell viability=live cell concentration/total cell concentration.

Another embodiment provides a method of determining the total live cell biomass of a cell aggregate composition comprising:
(a) contacting a cell aggregate composition with a dissociation enzyme to obtain a dissociated sample; and
(b) quantifying the number of single cells, cell clusters, and cell aggregates per mL in the dissociated sample by counting the number particles based on size, wherein the sum of biomass for each of the single cell population, cell cluster population, and cell aggregate population represents the total live cell biomass (LCB).

In some aspects, the biomass for each single cell population, cell cluster population, and cell aggregate population is calculated by obtaining the mean diameter of each population from a particle counting instrument, wherein biomass=4/3(π) r³.

In some aspects, counting the number particles based on size comprises counting single cells of particles 6-11 microns in diameter, cell clusters of particles 11-17 microns in diameter, and aggregates of particles more than 17 microns in diameter. In some aspects, the method further comprises determining the biomass per live cell by dividing the live single cell biomass concentration (i.e. the cubic volume per milliliter for particles of a given size ranges, for example 6-11 microns in diameter) by the live cell concentration (i.e., the number of particles per milliliter in the same size range) obtained by the present embodiments or aspects thereof. The biomass per cell may be calculated as Biomass (μm³) per cell=Single cell biomass (μm³) per ml/Number of single cells per ml.

In additional aspects, the method further comprises determining the number of live cells per milliliter, wherein Total live cell biomass (LCB) per ml/Biomass per Live cell=Live cells per ml. In some aspects, the method further comprises treating the dissociated sample with a nuclease prior to step (b). In some aspects, the method is performed in the absence of shear forces. In certain aspects, the method does not comprise trituration of the cell aggregate composition. In some aspects, the method further comprises determining the percent of viable biomass (VBM), wherein percent VBM=100×(LCB per mL/TCB per mL).

A further embodiment provides a method of determining the biomass of a cell aggregate composition comprising:
(a) measuring the size of each particle in the cell aggregate composition using a particle counting instrument, wherein the size is the diameter or radius of each particle in said cell aggregate composition; and
(b) obtaining a biomass of the cell aggregate composition, wherein biomass=4/3(π) r³.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The following drawings form part of the present specification and are included to further demonstrate certain aspects of the present invention. The invention may be better understood by reference to one or more of these drawings in combination with the detailed description of specific embodiments presented herein.

FIGS. 1A-1B: AO and PI fluorescent dyes classify live and dead cells using the CELLACA™™MX instrument. PRP aggregates were incubated with 10×TRYPLE™™ for 30 minutes at 37° C. A quench solution containing DNAase was added, and the sample was subjected to mechanical shear forces (e.g., 20 strokes of trituration through pipet tip) to singularize the cells. (FIG. 1A) The dissociated sample was incubated with a mixture of Acridine orange and propidium iodide (AOPI) and fluorescent images were collected using the CELLACA™™MX. (FIG. 1B) An image analysis algorithm was used to count live cells (green circles) and dead cells (red circles).

FIGS. 2A-2B: PRP aggregates were incubated with Solution 10 and triturated to prepare nuclei. (FIG. 2A) 50 ul aliquots were mixed with AOPI and analyzed on the CELLACA™™ MX. (FIG. 2B) All objects were labeled with PI and thus counted as dead cells by the CELLACA™™ software. The method provided a measure of the total number of cells.

FIGS. 3A-3B: Particle analysis with the MULTISIZER™4e instrument. Samples were analyzed using a 200 um aperture tube configuration and controlled settings for the sample volume (50 or 100 ul), analytic volume (2 ml) and electrolyte volume (100 ml) with stirring. (FIG. 3A) 50 ul bulk sample preparation of PRP aggregates (blue bars). A separate sample of PRP was completely dissociated and analyzed (red bars). To better visualize the size differences, particle diameters were converted to cubic volume (i.e., biomass) measurements and histograms were overlayed. The relative fraction of biomass in each data bin spanning 6 to 120 um particles is shown. (FIG. 3B) Separately, aggregates were incubated with Solution 10 and triturated to prepare nuclei as above. 100 ul of the nuclei prep was analyzed on the MULTISIZER™ 4e using the same instrument configuration. A histogram showing the number of particle counts per ml in data bins spanning 2 to 10 um particles is shown.

FIGS. 4A-4B: CELLACA™™ and Multisizer comparison studies. (FIG. 4A) After complete dissociation by TRYPLE™™ incubation (30 minutes at 37° C.) and trituration PRP samples (n=4 each) were analyzed on the Multisizer instrument. Singularized cells (e.g., 6.3 to 11 microns in diameter) were counted. In parallel, the triturated cells were labeled with AOPI and live cell counts were analyzed on the CELLACA™™ (n=6 wells each). (FIG. 4B) PRP aggregates were incubated with Solution 10 and triturated to prepare nuclei. 100 ul aliquots were analyzed on the MULTISIZER™ and designated 1-M to 4-M (on the x-axis). Objects with diameters of 2.8 to 4.8 were counted. 50 ul aliquots were mixed with AOPI and analyzed on the CELLACA™MX and designated as 1-C to 4-C (on the X-axis).

FIG. 5: Percent viability in PRP aggregates. 5 ul samples of aggregates were incubated in Solution 10 to lyse cells and prepare nuclei. Labeling on the x-axis denotes samples of various PRP lots: iPRP0045A, iPRP0028, iPRP0025A, iPRP0028, iPRP0055C. 50 or 100 ul samples were analyzed on the CELLACA™ or the Multisizer instruments to assess the total cell concentration. CELLACA™ samples were mixed with equal volumes of AOPI and nuclei were counted using the FL2 channel. The Multisizer counts were obtained by counting particles >2.8 microns in diameter. The corresponding live cell concentration values were divided by total cell concentration values to obtain a calculation for percent viability. This calculation was performed with separate sets of data from the CELLACA™ and Multisizer.

FIGS. 6A-6B: Disappearance of dead cells. (FIG. 6A) CELLACA™ data output following complete dissociation of PRP aggregates and AOPI labeling resulted in 90% of the counts categorized as viable cells. Large diffuse PI labeled (dead) cells are present in this field of view, as well as one smaller cell with a higher fluorescence intensity. (FIG. 6B) A separate dissociated sample was labeled with AOPI, added to three (3) separate wells of a CELLACA™ plate and PI+ cells were counted. The same wells were reanalyzed after 1, 2, or 15 minutes.

FIGS. 7A-7C: Disappearance of dead cells result in lower counts. PRP aggregates were incubated in 10×TRYPLE™™ for 30 min, quenched with a DNAase containing solution, triturated through a pipet tip, and then labeled with AOPI dye. 15 min after exposure to dye, 3-channel images were collected using the CELLACA™. (FIG. 7A) Digital magnification of a merged 2-channel fluorescent image is shown revealing a single (green) live cell in channel 1 (FL1) labeled with AO adjacent to two PI stained dead (red) cells in channel 2 (FL2). (FIG. 7B) The same three cells are represented using the brightfield channel merged with the FL2 channel revealing the absence of membranes surrounding the PI labeled cells. (FIG. 7C) The red and green circles on the same image indicate that the CELLACA™ counting algorithm successfully classified the AO-labeled cell as a live cell, but only one of dead cells was classified.

FIGS. 8A-8G: Room temperature incubation of aggregate samples in 10×TRYPLE™. 10-20 ul volumes of PRP0028 aggregates were incubated in 400 ul of 10×TRYPLE™ at room temperature for 2 minutes (FIGS. 8A-8B), 10 minutes (FIG. 8C-8D), or 30 minutes (FIGS. 8E-8G), then gently mixed with AOPI, avoiding excessive trituration. Exposure times for the fluorescent channel 1 (FL1) was 150 msec. Exposure times for fluorescent channel 2 (FL2) was 500 msec. FIGS. 8A, 8C, 8E, and 8F show merged 2-channel fluorescent images with live (green) cells labeled with acridine orange (AO) in FL1, and dead (red) cells labeled with propidium iodide (PI) in FL2. FIG. 8G shows only the FL2 channel. FIGS. 8B, and 8D show the brightfield (BF) channel merged with the FL2 channel.

FIGS. 9A-9F: Elimination of dead cells prior to complete dissociation of aggregates. PRP0029 aggregates were incubated with 10×TRYPLE™ at 37° C. for 2 minutes (FIGS. 9A-9B), 10 minutes (FIG. 9C), or 30 minutes (FIG. 9D), then gently mixed with AOPI, avoiding excessive trituration. Separately, the control method was employed which involved 20 strokes of trituration (FIGS. 9E-9F). After mixing the samples with AOPI, fluorescent images were collected using the CELLACA™ MX (as described in FIG. 9). FIG. 9B shows the brightfield (BF) channel merged with the FL2 channel. All other panels show 2-channel fluorescent images. FIG. 9E shows imaging of the control condition after trituration. These cells were labeled with AOPI and analyzed within 2 minutes. The image in FIG. 9F was captured from the same population of cells 15 minutes after the addition of AOPI.

FIG. 10: Particle size histograms of PRP before and after trituration. After incubation in 10×TRYPLE™ (30 minutes at 37° C.) a quenching reagent containing DNAase was added and incubated at room temperature until the suspension was clear. The sample was gently mixed and 100 ul was transferred to a 100 ml electrolyte solution with stirring and Multisizer analysis was carried out. Particle sizes were converted to cubic volume (biomass) measurements. The relative fraction of biomass in data bins spanning 3 to 25 um is shown. The dashed lines represent the gating parameters (6.3 to 11 microns) that define single cells.

FIGS. 11A-11B: Representative histograms. (FIG. 11A) number of counts per ml in a dissociated sample. (FIG. 11B) Biomass per ml in the same sample.

FIG. 12: Particle size histograms of 10×TRYPLE™ treated samples of PRP before and after trituration. A bulk sample of PRP was prepared. A total of 9×20 ul aliquots were transferred into 400 ul volumes of 10×TRYPLE™ and incubated for 30 min at 37° C. and then a quench reagent containing DNAas was added. The samples were divided into three sets. Set 1 (complete dissociation) was triturated 20 times and processed. Set 2 received one stroke of trituration and set 3 received no trituration. The samples were analyzed on the Multisizer analysis. The relative fraction of biomass in data bins spanning 2 to 60 um is shown. The dashed line represents a 6.3um threshold.

FIG. 13: PRP aggregate biomass with cell counter versus particle counter.

FIG. 14: Biomass correction calculations reveals similar cell concentrations in all conditions.

FIGS. 15A-15B: Multisizer histograms used to collect data for biomass correction calculations. (FIG. 15A) Calculating biomass/live cell. Biomass concentration gated from 6.3-11 microns to indicate biomass of only single cells. (FIG. 15B, left) Particulate count data gated from 6.3-11 microns to indicate number of only single cells. (FIG. 15B, right) Converting clusters to cell counts. Biomass concentration gated above 6.3 microns to indicate biomass of all cells in the sample.

FIG. 16: Percent viability calculated using cell counter method or present particle counter (e.g., Multisizer). By using 10×TRYPLE™™ to remove a significant portion of dead cells, viability was estimated. Particle counter to determine percent viability by biomass shows similar percent viability to the assay using cell counter with cell labeling.

FIG. 17: Differential volume of particles of different diameters. The red bars represent the 10×TRYPLE™™ dissociated (TD) sample, after quench and trituration. The TD sample is not 100% fully dissociated as shown by some aggregates between 20 and 40 μm. The present methods of determining biomass (e.g., >6 microns) would capture these aggregates while a cell labeling method (e.g., using CELLACA™ or Nucleocounter) would not be able to resolve how many cells are in the aggregates. The blue bars are the bulk non-dissociated aggregates, 20 μl dispensed directly into a Multisizer cup with a defined volume of Isoton. This lot has poor percent aggregate biomass, as evidenced by the blue peak around 6-10 microns which are mostly individualized dead cells. After the bulk prep analysis, 1×TRYPLE™ was added directly to the same Multisizer cup and reanalyzed within 60 seconds as shown by the purple bars. This immediately knocked down the dead cell peak. The addition of TRYPLE™ here effectively diluted the sample by 10%, so the concentration of aggregates, and thus the magnitude of the 33 μm aggregate peak was reduced. However, interestingly, the mean size of the aggregates was unchanged. There are at least two explanations here: 1) there were no dead cells in the 50A aggregates, or 2) the removal of dead cells did not change the overall diameter of the aggregates. A closer look at the Aggregate-gated volume stats (>17 microns) suggest the latter explanation. The loss in biomass after 60 seconds was 23%, not 10%. Finally, the same sample was incubated at room temperature with stirring in 1×TRYPLE™ for 30 minutes (green bars). In the final analysis, the aggregates remained the same size, but the amount of biomass was further reduced by an additional 30%. The amount of aggregate biomass remaining was 34.93/64.55=54%.

FIGS. 18A-18B: (FIG. 18A) DNA standard curve was performed with each assay to determine the fluorescence range of the assay. Samples were excluded from analysis if their fluorescence values did not fall within the DNA standard curve. (FIG. 18B) The linear range of the assay was between 2,500 and 20,000 cells per well for the lysed sample.

FIGS. 19A-19D: (FIG. 19A) Across a series of cellular concentrations per well from 1.70e4 to 1.36e5, and measuring viability using the above method (CellTox Green), an approximately similar value was reported for viability at close to 40%. This was slightly higher than the value recorded using the complete dissociation method (control or current best practice (CBP)) assay using the CELLACA™, and the percent CV for fluorescent values across well replicates was <10%. This assay was performed on PRP aggregates stored in a refrigerator (4° C.) overnight in balanced salt solution (BSS). (FIG. 19B) The assay was repeated using freshly thawed PRP aggregates. Similar consistency in viability was observed for a range of cell concentration per well, with percent CV under 20% for well-to-well replicates. Viability was higher using the CellTox assay, possibly due to using freshly thawed PRP aggregates. (FIG. 19C) To test assay proportionality across a viability range, cells were killed by storing PRP aggregates in BSS overnight at 37 C, and these were mixed in controlled ratios with cells stored overnight at 4 C. Cells at 4 C have some viable cells. Mixing cells at controlled ratios led to proportional viability measurements across the series. (FIG. 19D) When testing PRP as aggregates as aggregates and single cells, the assay was proportional across a range of concentrations. A new linear range of this assay was obtained with a high concentration of 20,000 cells/well.

FIGS. 20A-20C: (FIG. 20A) Percent viability was directly compared to the CELLACA™ using the new linear range, and viability was similar between operators and observed to be higher than viability recorded on the CELLACA™. (FIG. 20B) Viability across multiple operators was tested on the CellTox assay. All operators recorded higher viability than the CELLACA™, but there was some operator-to-operator variability. (FIG. 20C) Using PRPs, the CellTox Green assay was directly compared to the CELLACA™ and Multisizer. The CellTox Green assay recorded higher percent viability than other viability assays.

### I. DESCRIPTION OF ILLUSTRATIVE EMBODIMENTS

It is common for induced pluripotent stem cell (iPSC)-derived cells to be manufactured as aggregates. This is due to their biology, as cell to cell contact supports their viability, and a desire to utilize large scale bioreactors. Furthermore, iPSC-derived transplantable cells may be cryopreserved as 3D aggregates. While it is possible to count cells within aggregates using confocal imaging, these methods are very difficult to validate and require highly sophisticated and expensive tools. Therefore, the determination of cell concentration from aggregates in suspension typically requires a method to individualize cells prior to counting. This is typically achieved with dissociative enzymes (e.g., Trypsin) and the method may also include mechanical agitation to create shearing forces to separate cells.

Dose formulation for the clinical cell aggregate product, such as photoreceptor precursor cells, was studied using this approach. Briefly, aggregates were thawed and processed to create a high (i.e., bulk) concentration, and then small volume (e.g., 10 ul) samples were transferred into an enzyme solution (e.g., Accutase, trypsin, TRYPLE™ (a recombinant trypsin-like protease), or dispase) to dissociate aggregates and individualize the cells. Additional steps included treatment with DNAase and mechanical shearing through pipetting (i.e., trituration). Finally, the samples were analyzed using cell counters based on cell staining (e.g., Trypan Blue, acridine orange (AO)/propidium iodide (PI), Hoechst, 4′,6-diamidino-2-phenylindole (DAPI), Phycoerythrin (PE), Allophycocyanin (APC), Fluorescein isothiocyanate (FITC), Carboxyfluorescein diacetate (CFDA), Calcein AM, or 7-aminoactinomycin D (7AAD)), such as VICELL™ or CELLACA™® MX cell counters, the concentration of the dissociated cells was reported, and calculations were performed to estimate cell concentration in the bulk (i.e., non-dissociated) cell aggregates.

This method of enzyme treatment followed by trituration can be effective at dissociation, and therefore facilitates counting live cells. However, it is unclear how many viable cells die during this process. Specifically, trituration can damage cell membranes, allowing spurious dead cell labeling and misclassification and/or the elimination of viable cells. The latter effect may result in an under-estimation of the viable cell concentration. Conversely, it is also known that this method eliminates dead cells—more specifically cells that stain with dead cell dyes (i.e., propidium iodide or Trypan blue). This may result in an over-estimation of the viable cell fraction (i.e., percent viability).

To date, a method has not been identified to dissociate cell aggregates in a manner that preserves live cells without eliminating dead cells. For assessment of percent viability in cell aggregates, previous methods employ enzymes (e.g., 10×TRYPLE™) to dissociate live cells, and then perform a separate assay. For example, a sample taken from the same bulk preparation may be subjected to a cell lysis solution (e.g., Solution 10) for the preparation of cell nuclei which may be counted using an automated cell counter. In this manner, the concentration of nuclei may be used as a measure of all cells (i.e., live and dead). The dead cell count may be calculated by subtracting the live cell count from the total nuclei count, and the percent viability may be calculated by dividing the live cell concentration (e.g., TRYPLE™-dissociated cells) by the nuclei concentration.

This two-part assay method for cell enumeration and viability assessment of thawed cell aggregates has limitations for dose formulation. Most notably, the trituration step is a manual pipetting exercise that shears clusters of cells. However, the amount of shear force varies between users, and this may result in incomplete dissociation or, conversely, damage to live cells. Following incubation with live and/or dead cell dyes, the cell labeling pattern may change over time. This may be the result of cell toxicity, variables that affect the uptake of the dye, or instability of the fluorescence intensity. These variables, and others, may cause inaccurate cell concentration measurements. The lack of a robust counting assay has resulted in a lack of consistency in dose formulation of cell aggregates, such as PRP aggregates.

Accordingly, in certain embodiments, the present disclosure provides an assay method to improve the consistency of cell counting when the starting material are cell clusters and/or cell aggregates. The present methods can provide an accurate measure of both live and dead cell concentrations without the need to fully dissociate the cell aggregate samples. In particular aspects, the present methods comprise the elimination of dead cells without mechanical forces, such as trituration, which can make automated cell counting inaccurate due to the damage done to live cell membranes. The present methods can also avoid the use of labeling dyes.

Instead of counting dissociated cells, in certain embodiments, the present methods assess the three-dimensional volume (referred to here as “biomass”) occupied by individualized cells, cell clusters, cell aggregates, or any mixture of these entities. This may be accomplished by particle counting technology (e.g., Beckman Coulter MULTISIZER™ 4e Coulter Counter) which are based on movement of cells through the aperture in a glass tube or by image analysis using automated cell counting instruments which are image based on 2D labeling with an algorithm to determine the cell count. The MULTISIZER™ 4e uses the Coulter principle to detect particles via electrical zone sensing, regardless of the particle's nature or optical properties. In both cases, the size (e.g., diameter or radius) of objects is the primary unit of measure. With the assumption that cells are spheres, a simple mathematical transformation of object radius (r) may be performed where:

$$Biomass = Volume of a sphere = 4 / 3 ( π ) {r}^{3} \qquad Equation \#1$$

In samples that contain dissociated cells of known size, this calculation provides a reference value of cubic microns (μm³) per cell. Similarly, in samples that contain aggregates of known size, a value for cubic microns (μm³) per aggregate may be derived. Taken together, in theory, these two values provide an equation to calculate the number of live cells per aggregate, where:

$$Biomass per aggregate / Biomass per live cell = number of live cells per aggregate \qquad Equation \#2$$

In a first aspect, a small fraction of the cell aggregate composition (e.g., 1%) may be completely dissociated using an enzyme (e.g., TRYPLE™) followed by DNAase treatment and trituration. Then, a fixed volume (e.g., 0.5 mL) may be analyzed using a particle counting instrument (e.g., Multisizer4e) configuration appropriate for detection of single cells. The configuration may include the use of a 100 μm aperture tube and software settings that report the number of counts greater than 6.3 μm. The latter method has been shown to be useful for counting dissociated cells (e.g., PRP cells) and it excludes debris (e.g., small objects less than 6.3 μm). By controlling the sampling and assay volumes, the instrument may output the live cell concentration without the use of labeling dyes, and thus provide the necessary information to adjust the live cell concentration of the bulk aggregate to a target concentration for dose formulation.

In a second aspect, a small fraction of the cell aggregate composition (e.g., 1%) may be treated with a lysis solution (e.g., Solution 10, an acidic aqueous solution of surfactant and organic acid with a pH value in the range of 2.00-3.00, may comprise ammonium chloride, potassium carbonate, and EDTA) that disrupts the cytoplasmic membrane, but leaves the nuclear membrane intact. A defined volume (e.g., 0.5 mL) of the nuclei preparation may be analyzed using a particle counting instrument (e.g., Multisizer4e) configuration appropriate for detection of nuclei. The configuration may include the use of a 100 μm aperture tube and software settings that report the number of counts greater than 3.8 μm. The latter method has been shown to be useful for counting nuclei, and excludes debris (e.g., small objects less than 3.8 μm). By controlling the sampling and assay volumes, the instrument may output the total cell concentration without the use of labeling dyes.

Using one sample treated with a dissociation enzyme and a second sample treated with cell lysis reagent, the particle counter analysis may provide both the live cell concentration and the total cell concentration. The percent viability of the aggregate sample may therefore be obtained using the following equation:

$$Percent Viability = Live cell concentration / total cell concentration \qquad Equation \#3$$

In some aspects, the aggregate sample may be treated with TRYPLE™ to eliminate dead cells, followed by treatment with DNAase, but in the absence of shear forces (e.g., trituration). The particle counter (e.g., Multisizer) may be used to report a distribution of particle sizes in this population which may include single cells (e.g., particles 6-11 microns in diameter), cell clusters (e.g., particles 11-17 microns in diameter) and aggregates (e.g., particles >17 microns in diameter). By knowing the concentration of dissociated live cells (as described above) and the mean diameter of this population, the Biomass per live cell may be calculated by Equation #1. This value may be applied to the analysis of the incompletely dissociated cell clusters and aggregates, provided that the range of particle detection is adequate (for example, the 100 um aperture has a range of detection from 2 um to 60 μm). Within each bin of the size distribution output, the number of cells, cell clusters or cell aggregates per ml may be converted to Biomass per ml using Equation #1. The sum of biomass measurements for all three populations (e.g., all particles greater than 6.3 μm) represents the total live cell biomass (LCB).

Using the diameter of the live cell population as a constant value, the number of live cells/ml may be obtained using the following calculation:

$$Total live cell biomass ( LCB ) per ml / Biomass per Live cell = Live cells per ml \qquad Equation \#4$$

This method of establishing the live cell concentration from a suspension of aggregates enables a dose formulation strategy without the need to apply shearing forces which may damage cell membranes and lead to misclassification of dissociated cells.

In certain aspects, the present methods provide novel clinical cell therapy dosing metrics whereby the potency of a cell therapy drug product is measured in terms of total cellular biomass rather than dissociated cell numbers. The total cellular biomass (TCB) of the untreated cell aggregate (i.e., drug) product may be assessed using a particle counter and the appropriate instrument configuration. For example, after thawing and carrying out dose formulation, the PRP cell aggregate composition is comprised of aggregates (e.g., 30-60 microns in diameter) and a second population of dead cells (e.g., about 6 microns in diameter). Both populations may be analyzed using the particle counter, the appropriate instrument configuration may include the 200 um aperture tube which detects 4-120 μm particles.

With this approach, a defined volume (e.g., 50 ul) of the untreated cell aggregate (e.g., PRP cell aggregates) may be analyzed and the TCB/ml may be determined. Separately, a dissociated sample (as described above) may be collected and analyzed separately on the particle counter to obtain the LCB. Both metrics, TCB and LCB, may be informative regarding quality control metrics for clinical applications. Taken together, the two values also provide a new metric for aggregate product viability which is described here as the % of viable biomass (VBM). Conceptually, this metric is closely related to assessments of % cell viability following dissociation of aggregates, because it describes the fraction of the product that withstands TRYPLE™ treatment. The percent of VBM may be calculated as follows:

$$Percent VBM = 100 \times ( LCB per mL / TCB per mL ) \qquad Equation \#5$$

### I. DEFINITIONS

The term “purified” does not require absolute purity; rather, it is intended as a relative term. Thus, a purified population of cells is greater than about 90% 91%, 92%, 93%, 94%, 95%, 96%, 97%, 98%, 99% or 100% pure, or, most preferably, essentially free of other cell types.

As used herein in the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

As used herein, a composition or media that is “substantially free” of a specified substance or material contains ≤30%, ≤20%, ≤15%, more preferably ≤10%, even more preferably ≤5%, or most preferably ≤1% of the substance or material.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “cell population” is used herein to refer to a group of cells, typically of a common type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and depleted of one or more cell types.

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

The term “pluripotent” refers to the property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. A pluripotent stem cell is an embryonic stem cell derived from the inner cell mass of a blastocyst. In other embodiments, the pluripotent stem cell is an induced pluripotent stem cell derived by reprogramming somatic cells.

The term “differentiation” refers to the process by which an unspecialized cell becomes a more specialized type with changes in structural and/or functional properties. The mature cell typically has altered cellular structure and tissue-specific proteins.

As used herein, “undifferentiated” refers to cells that display characteristic markers and morphological characteristics of undifferentiated cells that clearly distinguish them from terminally differentiated cells of embryo or adult origin.

“Embryoid bodies (EBs)” are aggregates of pluripotent stem cells that can undergo differentiation into cells of the endoderm, mesoderm, and ectoderm germ layers. The spheroid structures form when pluripotent stem cells are allowed to aggregate under non-adherent culture conditions and thus form EBs in suspension.

An “isolated” cell has been substantially separated or purified from others cells in an organism or culture. Isolated cells can be, for example, at least 99%, at least 98% pure, at least 95% pure or at least 90% pure.

An “embryo” refers to a cellular mass obtained by one or more divisions of a zygote or an activated oocyte with an artificially reprogrammed nucleus.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as October 3/4), Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

An “allele” refers to one of two or more forms of a gene. Diploid organisms such as humans contain two copies of each chromosome, and thus carry one allele on each.

The term “homozygous” is defined as containing two of the same alleles at a particular locus. The term “heterozygous” refers to as containing two different alleles at a particular locus.

A “haplotype” refers to a combination of alleles at multiple loci along a single chromosome. A haplotype can be based upon a set of single-nucleotide polymorphisms (SNPs) on a single chromosome and/or the alleles in the major histocompatibility complex.

As used herein, the term “haplotype-matched” is defined as the cell (e.g. iPS cell) and the subject being treated share one or more major histocompatibility locus haplotypes. The haplotype of the subject can be readily determined using assays well known in the art. The haplotype-matched iPS cell can be autologous or allogeneic. The autologous cells which are grown in tissue culture and differentiated to PRP cells inherently are haplotype-matched to the subject.

“Substantially the same HLA type” indicates that the Human Leukocyte Antigen (HLA) type of donor matches with that of a patient to the extent that the transplanted cells, which have been obtained by inducing differentiation of iPSCs derived from the donor's somatic cells, can be engrafted when they are transplanted to the patient.

“Super donors” are referred to herein as individuals that are homozygous for certain MHC class I and II genes. These homozygous individuals can serve as super donors and their cells, including tissues and other materials comprising their cells, can be transplanted in individuals that are either homozygous or heterozygous for that haplotype. The super donor can be homozygous for the HLA-A, HLA-B, HLA-C, HLA-DR, HLA-DP or HLA-DQ locus/loci alleles, respectively.

“Feeder-free” or “feeder-independent” is used herein to refer to a culture supplemented with cytokines and growth factors (e.g., TGFβ, bFGF, LIF) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

For a medium, extracellular matrix, or culture system used with human cells, the term “Xeno-Free (XF)” refers to a condition in which the materials used are not of non-human animal-origin.

“Pre-confluent” refers to a cell culture in which the proportion of the culture surface which is covered by cells is about 60-80%. Usually, pre-confluent refers to a culture in which about 70% of the culture surface is covered by cells.

The term “retinal progenitor cells”, also called “retinal precursor cells” or “RPCs”, encompass cells which are competent for generating all cell types of the retina, including neural retina cells, such as rods, cones, photoreceptor precursor cells, as well as cells which can differentiate into RPE.

The term “neural retinal progenitors” or “NRPs” refers to cells which are restricted in their differentiation potential to neural retina cell types.

The term “photoreceptor” or “PR” cells refer to cells that are within the photoreceptor lineage (i.e., maturation) pathway, both before and after upregulation of expression of rhodopsin (rods) or any of the three cone opsins (cones), which encompasses both early and late markers of photoreceptor cells (rod, cone or both).

The terms “photoreceptor precursor cells” or “PRP” refer to cells differentiated from embryonic stem cells or induced pluripotent stem cells which can differentiate into photoreceptor cells that expresses the cell marker rhodopsin or any of the three cone opsins. The photoreceptors may be rod and/or cone photoreceptors.

“Retinal pigment epithelium” refers to a layer of pigmented cells between the choroid, a layer filled with blood vessels, and the neural retina.

The term “retinal degeneration-related disease” is intended to refer to any disease resulting from innate or postnatal retinal degeneration or abnormalities. Examples of retinal degeneration-related diseases include retinal dysplasia, retinal degeneration, age-related macular degeneration, Stargardt disease, Best disease, choroideremia, inherited macular degeneration, myopic degeneration, RPE tears, macular hole, diabetic retinopathy, retinitis pigmentosa, inherited retinal disease or degeneration, inherited macular degeneration, cone-rod dystrophy, rod-cone dystrophy, congenital retinal dystrophy, Leber congenital amaurosis, retinal detachment, and retinal trauma.

A “therapeutically effective amount” used herein refers to the amount of a compound that, when administered to a subject for treatment of a disease or condition, is sufficient to affect such treatment.

“Mature” RPE cells are referred to herein as RPE cells which have downregulated expression of immature RPE markers such as Pax6 and upregulated expression of mature RPE markers such as RPE65.

RPE cell “maturation” refers herein to the process by which RPE developmental pathways are modulated to generate mature RPE cells. For example, modulation of cilia function can result in RPE maturation.

As used herein, the term “biomass” refers to the three-dimensional volume occupied by individualized cells, cell clusters, cell aggregates, or any mixture of these entities.

As used herein, the term “cell clusters” refers to particles 11-17 microns in diameter. Cell clusters can be seen completely in a microscope in the xy plane.

As used herein, the term “aggregates”, “cell aggregates” or “cell aggregate” composition refers to particles more than 17 microns in diameter. Aggregates can be seen in three dimensions (i.e., xyz) in a microscope.

As used herein, a “fraction” or “sample” refers to a subset of a larger composition for performing the assay or method. The fraction may comprise a specific percentage of the larger composition, such as 1%, 2%, 3%, 4%, 5%, etc. or a specific volume of the larger composition, such as 1 mL, 2 mL, 3 mL, 4 mL, 5 mL, etc.

As used herein, the term “dissociation” refers to separation of a composition comprising cell clusters and/or cell aggregates into a substantially or essentially single cell composition. The “dissociated” composition or sample may comprise a small percentage (e.g., 0.1%, 0.5%, 1%, 2%, 3%, 4%, 5%, etc.) of cell clusters and/or cell aggregates depending on the method of dissociation performed, such as contact with a dissociation enzyme, DNAse and/or the use of shear forces, such as trituration, agitation, or vortexing.

As used herein, the term “completely dissociated” refers to a population of cells that are singularized to a sufficient extent that the human eye or an image analysis algorithm can separate and count all the cells in a field of view.

A “particle counter” refers to a device which involves the measurement of impedance fluctuations between two electrodes as particles (e.g., single cells, clusters or aggregates) pass through an aperture. Exemplary particle counting devices include but are not limited to the Beckman Multisizer and VWR ORFLO MoxiZ®.

### II. CELL AGGREGATE COMPOSITION

In specific aspects, the present methods comprise methods for determining cell viability or accurate dose of a cell aggregate composition, such as PSC-derived cell aggregates. The PSCs may be human PSC (hPSCs), are embryonic stem cells (ESCs), tissue specific progenitor stem cells (TSPSCs), mesenchymal stem cells (MSCs), umbilical cord stem cells (UCSCs), bone marrow stem cells (BMSCs), or induced pluripotent stem cells (iPSCs),

PSCs may comprise embryonic stem cells (ESCs) or induced pluripotent stem cells (IPSCs) which have the ability to differentiate into several cell types that can be used in drug testing and also in the study and treatment of diseases.

iPSCs are artificial stem cells produced from somatic cells through co-expression of defined pluripotency-associated factors. Like embryonic stem cells (ESCs), they can typically proliferate and self-renew indefinitely in vitro and differentiate into derivatives of all three primary germ layers (i.e., ectoderm, mesoderm, and endoderm) as well as germ cells that give rise to the gametes. The basic paradigm in the use of PSCs for cell therapy purposes is that they are first differentiated into the desired cell types of interest, and the resulting specialized tissue-specific cells are then transplanted as cell suspensions or more complex tissue constructs into patients.

In 2006, Takahashi and Yamanaka discovered that mouse embryonic and adult fibroblasts could be reprogrammed to cells with the characteristics of ESCs by overexpression of a defined set of ESC-enriched transcription factors (Oct4, Sox2, Klf4, and c-Myc). The resulting cells, termed iPSCs, display infinite self-renewal ability (stemness) and can differentiate into all three embryonic germ layers (pluripotency). Human iPSCs are a promising prospect for cell therapy in a wide range of diseases for which there are currently no cures or effective therapies, such as neurodegenerative diseases of the central nervous system, heart infarction, diabetes mellitus, and diseases of the liver, lung, and kidney. Given that iPSCs can be produced in a patient-specific manner, they may be used in autologous transplantation,

In some aspects, PSCs may be genetically engineered to knock-out and/or knock-in a gene. This may be performed by various genome-editing approaches, including CRISPR technology, zinc finger nuclease (ZEN) and transcription activator-like effector nuclease (TALEN) technologies. In some aspects, the gene may be knocked-in to induce driving the transgene of endogenous genes, such as a promoter, or by engineering & fusion or peptide cleavage site tethered to the transgene.

PSCs may be differentiated into any of the 216 cell types found in an adult organism, such as neurons, cardiomyocytes, smooth muscle cells, osteocytes, hepatocytes, keratinocytes, insulin-producing cells, hematopoietic cells, and endothelial cells. In particular aspects, the present PSC-derived cells may comprise one or more of the 216 cell types. In some aspects, the transplanted engineered stem cell-derived tissue or organ can be made up of multiple cell types. In some aspects, the PSC-derived cells are photoreceptor cells, photoreceptor precursors, retinal epithelial cells, retinal progenitor cells, cardiomyocytes, endothelial cells, hepatocytes, retinal ganglion cells, neurons, chondrocytes, skin cells, or other cell types that may be used for cell therapy.

The major cell types used from the endoderm include hepatic cells and insulin-producing cells. The mesodermal progenitors obtained from ESCs and iPSCs includes cardiomyocytes, endothelial cells, and hematopoietic cells. These cell types could be used for treatment of ischemic heart disease, repair of ischemic tissue, and to obtain all types of blood cells, respectively. The cells differentiated into the ectoderm lineage include cells of the epidermis, external sense organs, and central and peripheral nervous system, such as functional neurons that can be used for the treatment of neurodegenerative diseases, such as acute spinal cord injury.

The induction of ESC and iPSC differentiation to produce different cell types requires complex differentiation steps with specific culture medium and growth factors, addition of cytokines, and supplements. The high differentiation potential of ESCs into specific cell lineages through in vitro systems, EB formation, or co-culture with stromal cells represents a source of cells that can be used for testing new drugs and also for cell therapy clinical trials.

With the exception of germ cells, any somatic cell can be used as a starting point for iPSCs. For example, cell types could be keratinocytes, fibroblasts, hematopoietic cells, mesenchymal cells, liver cells, or stomach cells. T cells may also be used as a source of somatic cells for reprogramming (U.S. Pat. No. 8,741,648). There is no limitation on the degree of cell differentiation or the age of an animal from which cells are collected; even undifferentiated progenitor cells (including somatic stem cells) and finally differentiated mature cells can be used as sources of somatic cells in the methods disclosed herein. iPSCs can be grown under conditions that are known to differentiate human ES cells into specific cell types, and express human ES cell markers including: SSEA-1, SSEA-3, SSEA-4, TRA-1-60, and TRA-1-81.

Major Histocompatibility Complex (MHC) is the main cause of immune-rejection of allogeneic organ transplants. There are three major class I MHC haplotypes (A, B, and C) and three major MHC class II haplotypes (DR, DP, and DQ).

MHC compatibility between a donor and a recipient increases significantly if the donor cells are HLA homozygous, i.e. contain identical alleles for each antigen-presenting protein. Most individuals are heterozygous for MHC class I and II genes, but certain individuals are homozygous for these genes. These homozygous individuals can serve as super donors, and grafts generated from their cells can be transplanted in all individuals that are either homozygous or heterozygous for that haplotype. Furthermore, if homozygous donor cells have a haplotype found in high frequency in a population, these cells may have application in transplantation therapies for a large number of individuals.

Accordingly, the iPSCs can be produced from somatic cells of the subject to be treated, or another subject with the same or substantially the same HLA type as that of the patient. In one case, the major HLAs (e.g., the three major loci of HLA-A, HLA-B and HLA-DR) of the donor are identical to the major HLAs of the recipient. In some cases, the somatic cell donor may be a super donor; thus, iPSCs derived from a MHC homozygous super donor may be used to generate PR/PRP cells. Thus, the iPSCs derived from a super donor may be transplanted in subjects that are either homozygous or heterozygous for that haplotype. For example, the iPSCs can be homozygous at two HLA alleles such as HLA-A and HLA-B. As such, iPSCs produced from super donors can be used in the methods disclosed herein, to produce PR/PRP cells that can potentially “match” a large number of potential recipients.

In some aspects, the present methods comprise cell aggregate compositions comprising retinal cell aggregates, such as RPE cell aggregates produced from iPSCs, such as by the method disclosed in PCT/US2016/050543 and PCT/US2016/050554, incorporated by reference herein in their entirety. In other aspects, the cell aggregate composition comprise photoreceptor or PRP cell aggregates produced by the methods disclosed in WO2019/204817, incorporated herein by reference in its entirety. The present methods may be applied to cell aggregates comprising RPE and/or PRPs, such as disclosed in WO2021/243203 or WO2021/243265, incorporated herein by reference in their entirety.

The cell aggregate composition may generally be seeded in an appropriate culture vessel, such as a tissue culture plate, such as a flask, multi-layer flask, 6-well, 12-well, 24-well, 96-well or 10 cm plate. A culture vessel used for culturing the cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CELLSTACK® Chambers, culture bag, and roller bottle, as long as it is capable of culturing the stem cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system ex vivo that supports a biologically active environment such that cells can be propagated. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

Cell aggregates can be cultured with the nutrients necessary to support the growth of each specific population of cells. Generally, the cells are cultured in growth media and a buffer to maintain pH. The medium can also contain fatty acids or lipids, amino acids (such as non-essential amino acids), vitamin(s), growth factors, cytokines, antioxidant substances, pyruvic acid, buffering agents, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM), Alpha MEM, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the cell aggregates are cultured in a fully defined and feeder free media.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 1′-thioglycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco).

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

In certain aspects, the cell aggregates are dissociated by incubation with a cell dissociation solution or enzyme, such as exemplified by Versene, Trypsin, ACCUTASE™ or TRYPLE™™. Cell aggregates can also be dissociated into an essentially single cell suspension by pipetting or trituration.

In addition, Blebbistatin (e.g., about 2.5 μM) can be added to the medium to increase cell aggregate survival after dissociation into single cells while the cells are not adhered to a culture vessel. A ROCK inhibitor instead of Blebbistatin may alternatively be used to increase cell aggregate survival after dissociation into single cells.

Once a single cell suspension of cell aggregates is obtained, the cells are generally seeded in an appropriate culture vessel, such as a tissue culture plate, such as a flask, multi-layer flask, 6-well, 12-well, 24-well, 96-well or 10 cm plate. A culture vessel used for culturing the cell(s) can include, but is particularly not limited to: flask, flask for tissue culture, dish, Petri dish, dish for tissue culture, multi dish, micro plate, micro-well plate, multi plate, multi-well plate, micro slide, chamber slide, tube, tray, CELLSTACK® Chambers, culture bag, and roller bottle, as long as it is capable of culturing the stem cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system ex vivo that supports a biologically active environment such that cells can be propagated. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The cells may be cultured on culture plates coated by one or more cellular adhesion proteins to promote cellular adhesion while maintaining cell viability. For example, preferred cellular adhesion proteins include extracellular matrix proteins such as vitronectin, laminin, collagen, and/or fibronectin, which may be used to coat a culturing surface as a means of providing a solid support for pluripotent cell growth. The term “extracellular matrix (ECM)” is recognized in the art. Its components can include, but are not limited to, one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other ECM components may include synthetic peptides for adhesion (e.g., RGD or IKVAV motifs), synthetic hydrogels (e.g., PEG, PLGA, etc.) or natural hydrogels, such as alginate.

The extracellular matrix proteins may be of natural origin and purified from human or animal tissues or, alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition is xeno-free. For example, in the xeno-free matrix to culture human cells, matrix components of human origin may be used, wherein any non-human animal components may be excluded.

In some aspects, the total protein concentration in the matrix composition may be about 1 ng/ml to about 1 mg/mL. In some preferred embodiments, the total protein concentration in the matrix composition is about 1 μg/mL to about 300 μg/mL. In more preferred embodiments, the total protein concentration in the matrix composition is about 5 μg/mL to about 200 μg/mL.

The cell aggregates can be cryopreserved, see for example, PCT Publication No. 2012/149484 A2, which is incorporated by reference herein. The cells can be cryopreserved with or without a substrate. In several embodiments, the storage temperature ranges from about −50° C. to about −60° C., about −60° C. to about −70° C., about −70° C. to about −80° C., about −80° C. to about −90° C., about −90° C. to about −100° C., and overlapping ranges thereof. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored 48 hours to about one week. In yet other embodiments, the cells are stored for about 1, 2, 3, 4, 5, 6, 7, or 8 weeks. In further embodiments, the cells are stored for 1, 2, 3, 4, 5, 67, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80, serum albumin, such as human or bovine serum albumin. In certain embodiments, the solution comprises about 1%, about 1.5%, about 2%, about 2.5%, about 3%, about 4%, about 5%, about 6%, about 7% ·, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8% · to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C. minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C. In particular aspects, the cells may be cultured on any suitable culture surface, particularly a culture surface permissible for transplantation, such as on a scaffold in GMP-compliant conditions. In particular aspects, the cells are cultured on ECM-, such as vitronectin-, coated surfaces, such as a multi-well plate (e.g., 6-well, 12-well, 24-well, 48-well, or 96-well) or a polymer-, such as poly (lactic-co-glycolic acid) (PLGA)-, coated scaffold on a transwell support, such as a snapwell insert. The cells may alternatively be cultured on collagen or laminin. In specific aspects, the culture surface is coated with a high concentration of vitronectin, such as more than 1 μg/cm², particularly 2 μg/cm², 3 μg/cm², 4 μg/cm², 5 μg/cm², 6 μg/cm², 7 μg/cm², 8 μg/cm², 9 μg/cm², 10 μg/cm², or more. In particular aspects, the media is serum-free or defined media and may comprise knockout serum replacement. The cells may be cultured at a density of 100,000 cells/cm² to 500,000 cells/cm², such as 150,000 cells/cm², 200,000 cells/cm², 250,000 cells/cm², 300,000 cells/cm², or 350,000 cells/cm², particularly about 300,000 cells/cm².

The present cell aggregate compositions may be used for transplantation such as cell rescue therapy or whole tissue replacement therapy. Certain embodiments can provide use of retinal cell culture to enhance ocular tissue maintenance and repair for any condition in need thereof, including retinal degeneration or significant injury. Retinal degeneration may be associated with age-related macular degeneration (AMD), inherited macular degenerations, Stargardt's macular dystrophy, Best disease, choroideremia, inherited retinal degenerations (including retinitis pigmentosa, cone/rod and rod/cone dystrophies), diabetic retinopathy, retinal vascular disease, damage caused by retinopathy pf prematurity (ROP), viral infection of the eye, and other retinal/ocular diseases or injuries/trauma.

The cell aggregate composition described herein, or a pharmaceutical composition including the cell aggregates, can be used for the manufacture of a medicament to treat a condition in a patient in need thereof. The cells can be previously cryopreserved. In certain aspects, the disclosed present cell aggregates are derived from iPSCs, and thus can be used to provide “personalized medicine” for patients with eye diseases. Alternatively, iPSCs generated from a healthy donor or from HLA homozygous “super-donors” can be used.

Various eye conditions may be treated or prevented by the introduction of the cell aggregate compositions clinical doses obtained using the methods disclosed herein. The conditions include retinal diseases or disorders generally associated with retinal dysfunction or degradation, retinal injury, and/or loss of retinal pigment epithelium and/or photoreceptors. Conditions that can be treated include, without limitation, degenerative diseases of the retina, such as Stargardt's macular dystrophy, retinitis pigmentosa, rod/cone and cone/rod dystrophies, macular degeneration (such as age-related macular degeneration, myopic macular degeneration, or other acquired or inherited macular degenerations), retinal damage caused by retinopathy of prematurity (ROP) and diabetic retinopathy. Additional conditions include Lebers congenital amaurosis, hereditary or acquired macular or retinal degenerations, Best disease, retinal detachment, gyrate atrophy, choroideremia, pattern dystrophy, other dystrophies of photoreceptor cells, and retinal damage due to damage caused by any one of photic, laser, inflammatory, infectious, radiation, neovascular or traumatic injury.

Pharmaceutical compositions of the cell aggregates clinical doses obtained by the methods disclosed herein are also provided. These compositions can include at least about 1×10³ cells, about 1×10⁴ cells, about 1×10⁵ cells, about 1×10⁶ cells, about 1×10⁷ cells, about 1×10⁸ cells, or about 1×10⁹ cells. In certain embodiments, the compositions are substantially purified preparations. Compositions are also provided that include a scaffold, a polymeric carrier and/or an extracellular matrix, and an effective amount of the cells quantified by the methods disclosed herein. The matrix material is generally physiologically acceptable and suitable for use in in vivo applications. For example, the physiologically acceptable materials include, but are not limited to, solid matrix materials that are absorbable and/or non-absorbable, such as small intestine submucosa (SIS), crosslinked or non-crosslinked alginate, hydrocolloid, foams, collagen gel, collagen sponge, polyglycolic acid (PGA) mesh, fleeces and bioadhesives.

Suitable polymeric carriers also include porous meshes or sponges formed of synthethic or natural polymers, as well as polymer solutions. For example, the matrix is a polymeric mesh or sponge, or a polymeric hydrogel. Natural polymers that can be used include proteins such as collagen, albumin, and fibrin; and polysaccharides such as alginate and polymers of hyaluronic acid. Synthetic polymers include both biodegradable and non-biodegradable polymers. For example, biodegradable polymers include polymers of hydroxy acids such as polyactic acid (PLA), polyglycolic acid (PGA) and polylactic acid-glycolic acid (PGLA), polyorthoesters, polyanhydrides, polyphosphazenes, and combinations thereof. Non-biodegradable polymers include polyacrylates, polymethacrylates, ethylene vinyl acetate, and polyvinyl alcohols.

Polymers that can form ionic or covalently crosslinked hydrogels which are malleable can be used. A hydrogel is a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are crosslinked ionically, or block copolymers such as PLURON1CS™ or TETRON1CS™, polyethylene oxide-polypropylene glycol block copolymers which are crosslinked by temperature or H, respectively. Other materials include proteins such as fibrin, polymers such as polyvinylpyrrolidone, hyaluronic acid and collagen.

The pharmaceutical compositions can be optionally packaged in a suitable container with written instructions for a desired purpose, such as the reconstitution of photoreceptor function to improve a disease or abnormality of the retinal tissue. In some embodiments, the photoreceptors produced by the disclosed methods may be used to replace degenerated photoreceptor cells of a subject in need therein.

### III. DETERMINATION OF VIABILITY OF CELL AGGREGATE COMPOSITION

Certain embodiments of the present disclosure concern determining the concentration of live cells, concentration of total cells, distribution of particles of a given size (e.g., population of single cells, cells cluster, and/or cell aggregates), determination of live cell biomass, total biomass, or biomass of a specific cell population in a cell aggregate composition, such as to determine an accurate percent viability of a cell aggregate composition. In particular aspects, the clinical dose of the cell aggregate composition can be determined without cell labeling and/or the use of shear forces, such as trituration, on the cell aggregate composition.

The cell aggregate composition may comprise single cells, cell clusters, and/or cell aggregates. The cell aggregate composition may comprise live cells, dead cells, and/or cell debris.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Determination of Aggregate Biomass

Generally, the method to dissociate PRP aggregates comprises incubating the cells for 30 minutes in 10×TRYPLE™™ at 37° C. TRYPLE™™ is a highly stable form of Trypsin which cleaves cell surface proteins without disrupting the cytoplasmic membranes of viable cells. However, the membranes of dead cells may be disrupted by TRYPLE™™. This has practical consequences for efforts to dissociate freshly thawed PRP aggregates because there are significant numbers of dead cells present at thaw. This is a feature common to many cryopreserved iPSC-derived cell types. The activity of TRYPLE™™ is believed is believed to cause the breakdown of dead cells and the release of DNA which in turn causes the formation of a flocculant. To eliminate the flocculant, a quenching reagent can be added which includes DNAase (e.g., Benzonase). The working volumes typically include 400 ul of 10×TRYPLE™™, a 10-20 ul of the aggregate sample, and a volume of the quenching reagent (e.g., about 780-790 ul) that adjust the final volume to 1.2 ml.

To ensure complete dissociation, the final step in the dissociation protocol calls for the use of mechanical shear forces. This is accomplished by pipetting aggressively 20 strokes through a P1000 pipet tip (i.e., trituration). Without the trituration step, live cells may remain in aggregate form or in small clusters, making it difficult to use automated cell counting algorithms.

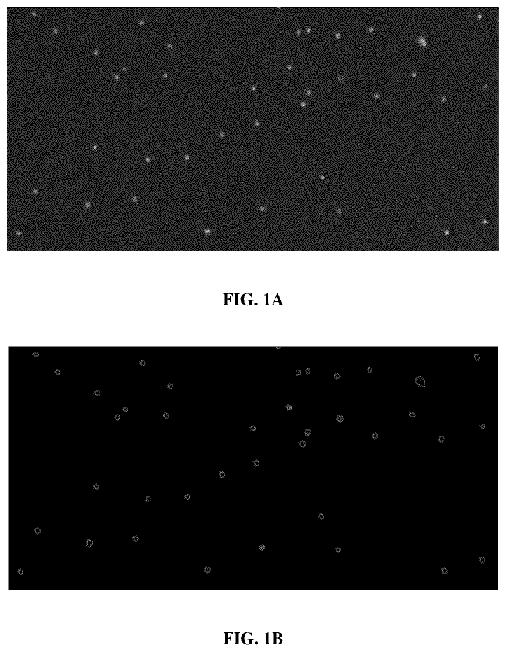

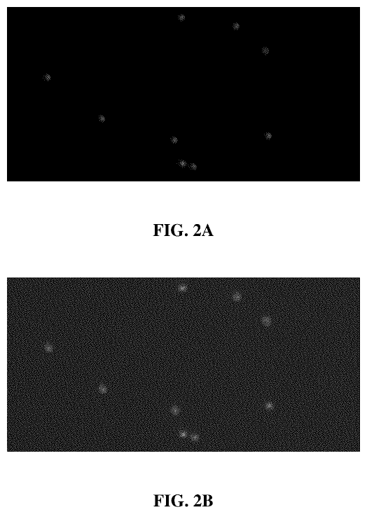

Automated cell counters make use of image analysis algorithms to count singularized live and dead cells with the use of labeling dyes. For example, the CELLACA™ MX platform uses acridine orange (AO) and propidium iodide (PI) to label live and dead cells respectively, and provide fluorescence based image analysis. The accuracy of this method for the classification of live and dead cells relies on the exclusion of propidium iodide by live cells that have intact cytoplasmic membranes. The algorithm classifies AO labeled cells (emitting green fluorescence in the FL1 channel) as live cells, and PI labeled cells (emitting red fluorescence in the FL2 channel) as dead cells (FIG. 1). In this manner, the live and dead cell concentrations may be obtained provided.

Because the method for dissociation of PRP aggregates causes the disappearance of dead cells, the data output from the CELLACA™ algorithm assigns a low dead cell count and therefore an artificially high value for the percentage of live cells (i.e., percent viability).

Several alternative dissociation methods have been considered, however, to date, none have proven successful for the preservation of the dead cell population. This fact has limited the utility of automated cell counters in assigning viability specifications to iPSC-derived aggregates, such as PRP aggregates. The CELLACA™ and ViCell algorithms are currently used to define only the live cell concentration for PRP aggregates.

Thus, it was necessary to develop a separate method to assess the concentration of all cells (i.e., live plus dead cells) present in PRP aggregates. The latter method involves the use of a low pH reagent (e.g., Solution 10) to lyse the cytoplasmic membranes, followed by a trituration step, to prepare a suspension of nuclei. Nuclei are subsequently labeled with AOPI. This method typically results in a homogeneous completely dissociated population of PI labeled nuclei with no AO labeled cells. The CELLACA™ counting algorithm may be applied for counting all nuclei to provide a measure of the total cell concentration (FIG. 2).

Particle counting using Coulter technology was considered as an alternative to the use of automated cell counters. Coulter counting instruments (Beckman MULTISIZER™, VWR ORFLO MOXIZ®) involves the measurement of impedance fluctuations between two electrodes as particles (e.g., single cells, clusters, or aggregates) pass through an aperture.

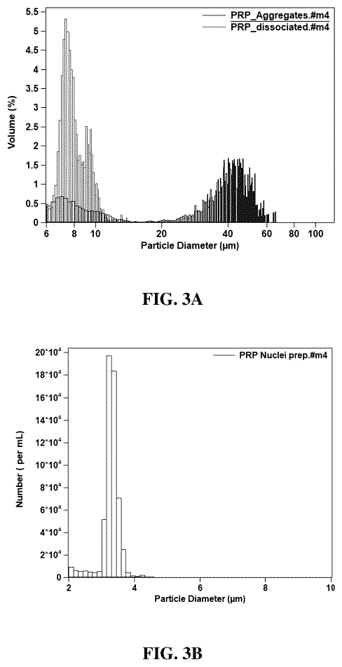

Comparative studies were carried out to determine if the Beckman MULTISIZER™ 4e particle counter would produce similar counts of dissociated cells as well as nuclei counts. The method described above for complete dissociation of PRP aggregates was performed to prepare singularized cells. Separately, the Solution 10 lysis method was carried out to prepare nuclei. Samples from each process were analyzed on the MULTISIZER™ (FIG. 3).

The untreated PRP aggregates (FIG. 3A, blue bars) were comprised of two peaks; aggregates of approximately 30-50 um in size, and single cells approximately 6-8 um in size. After the complete dissociation method (FIG. 3A, red bars), the aggregates were reduced to two smaller peaks approximately 7 and 9 um in size (red bars). This profile is consistent with the presence of photoreceptor rods and cones, the two main cell types present in the PRP product. There were very few particles larger than 11 microns, an indication that dissociation was complete. Also, it was noted that the diameter measurements obtained from the MULTISIZER™ were comparable to those obtained from singularized cells using the CELLACA™ algorithm to analyze images. The nuclei sample produced a relatively homogeneous distribution of particle sizes between 3 and 4 um in size (FIG. 3B). These observations suggested that it would be possible to use the particle counting method in combination with the method of complete dissociation to count singularized cells in the range of 6.3 to 11 microns. Similarly, it would be possible to perform particle counting in the range of 2.8-4.5 microns to assess the total cell counts from a nuclei preparation.

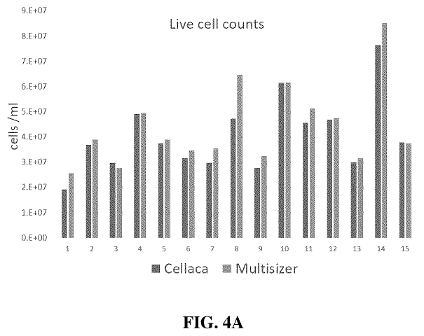

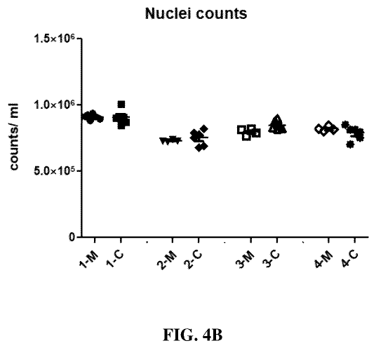

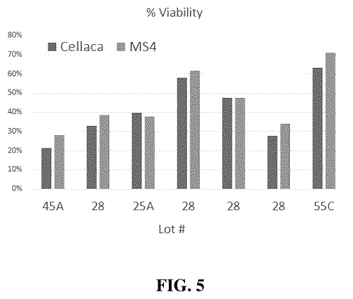

A comparison of AO labeled live cells counted on the CELLACA™ and particle counts from singularized (i.e., non-labeled) PRP cells was carried out across 15 different PRP samples, representing 7 different manufactured lots (FIG. 4). On average, there were 10% higher counts obtained from TRYPLE™™-dissociated samples using the MULTISIZER™ particle counting method.

Separately, nuclei preparations were made from four different PRP aggregate samples and a comparison was made between PI labeled counts on the CELLACA™ and particle counts on the MULTISIZER™. The nuclei counts were very similar between the two instruments. On average, there were 1.3% higher counts obtained from the CELLACA™, however the differences were insignificant.

Nuclei were prepared from a subset of the PRP aggregates in FIG. 4A, and the percent viability was determined (FIG. 5). On average, the Multisizer predicted a 13% higher viability in the same PRP samples (FIG. 5). This was reminiscent of the observation in FIG. 4A. It was concluded that the CELLACA™ data produced lower calculated values for percent viability because this instrument generated lower viable cell counts.

Acridine orange (AO) labels all cells, including dead cells. But in the presence of both AO and PI, the CELLACA™ algorithm will classify a double labeled cell as a dead cell. Conversely, the Multisizer does not differentiate between live cells and those with compromised cell membranes (e.g., dead cells) which may be of similar same size. Thus, it was sought to examine whether the differences observed between the CELLACA™ counts and the Multisizer counts could be explained by the presence of dead cells in the completely dissociated samples.

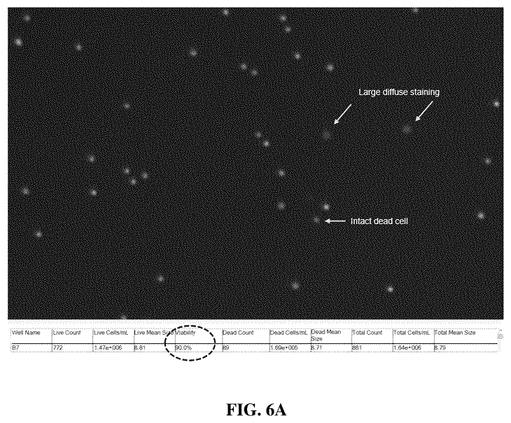

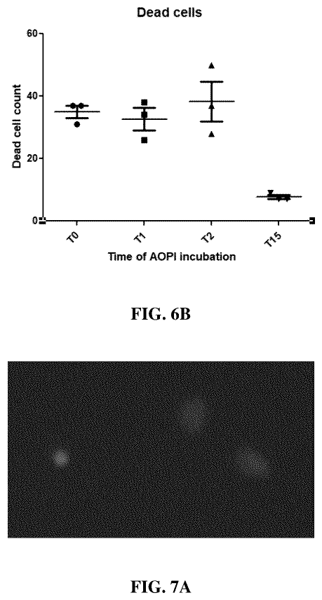

The data set from FIG. 4A was re-examined to look for the presence of dead cells in the CELLACA™ counts. When the total cell count (i.e., live cells plus dead cells) was compared to the Multisizer counts, there was improved comparability. However, the number of dead cells present in each set of data varied greatly. In some samples, dead cells accounted for 10% of the total cell count. In others, dead cells were entirely absent. Furthermore, when dead cells were present in the dissociated sample, it was noted that the size and PI staining intensity was variable (FIG. 6). In general, PI labeled cells were either small and brightly labeled or large and dim. Anecdotally, it was noted that if dissociated samples were analyzed soon after the AOPI labeling step, and then re-analyzed 15 minutes later, the dead cell population was greatly reduced. This prompted an experiment to determine if there was a dead cell population in the dissociated sample that was unstable.

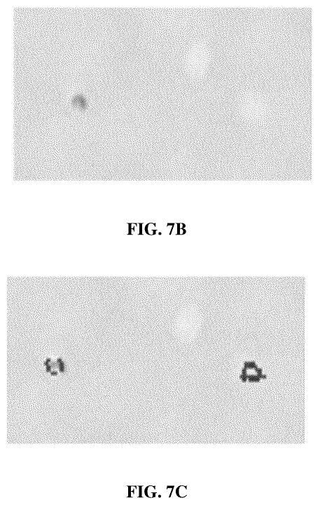

The results were consistent with anecdotal reports from other CELLACA™ users and suggested that the dead cells in the dissociated samples disappeared within 15 minutes of the addition of AOPI. Upon closer examination of the dead cells that remained after 15 min, it was noted that the brightfield images showed the absence of cell membranes. Dead cells appeared to disintegrate before losing PI+ staining intensity and disappearing altogether. (FIG. 7).

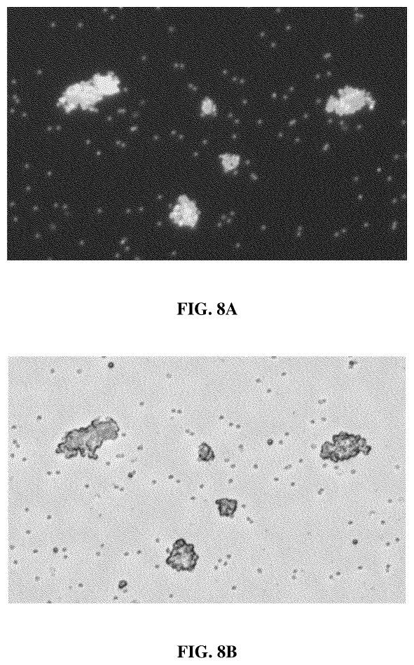

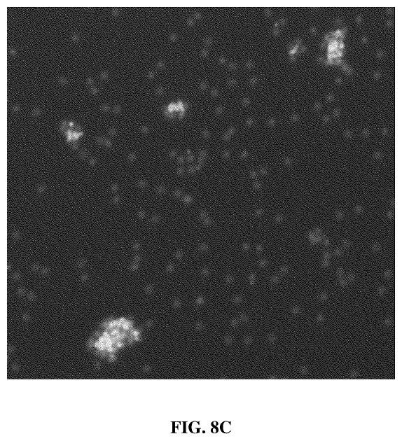

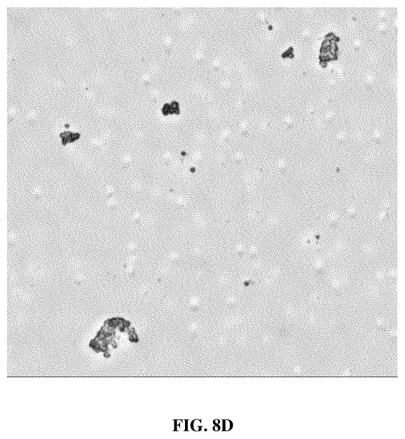

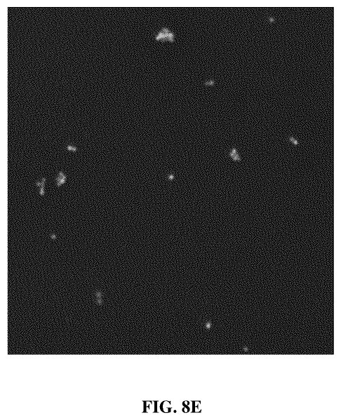

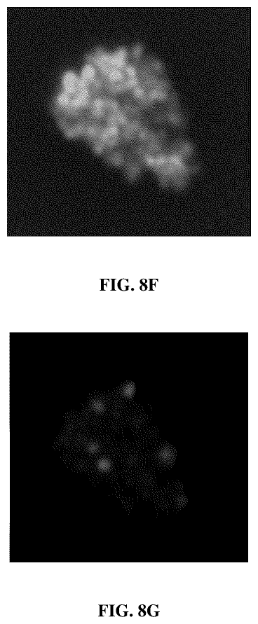

These observations provided an explanation for the variability observed in the dead cell counts. The time that elapsed after the addition of the AOPI reagent was not tightly controlled in the experiments represented in FIGS. 4 and 5. In samples analyzed promptly, the dead cell count may have comprised 10% of the total count. If there was a delay, dead cells may have disintegrated due to the presence of TRYPLE™, and the PI labeled DNA may have been degraded by residual DNAase. It should also be noted that this analysis would not invalidate the aforementioned experiments because only live cell counts were reported, and the live cell (AO labeled) population in the dissociated samples is much more stable. These observations also offered an explanation for the 10% discrepancy between the Multisizer and CELLACA™ counts. The Multisizer counts may have included the transient unstable population of cells described above. This line of reasoning would suggest that the Multisizer provided an inaccurate assessment of the percent viability by including dead cells while counting dissociated cells. However, this requires an assumption that dead cells remain present after the 30-minute incubation in 10×TRYPLE™. It was sought to test this idea by looking for the presence or absence of dead cells during the CBP process. A time course analysis was carried out and fluorescent images of PRP aggregates were captured following incubation in 10×TRYPLE™ (FIG. 8).

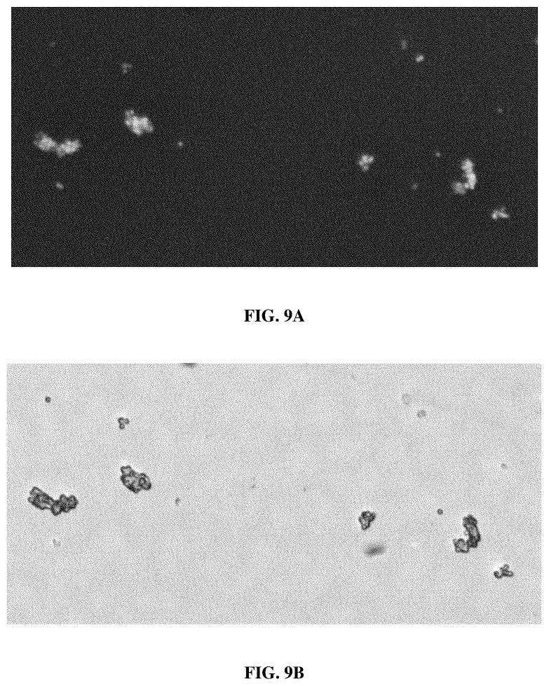

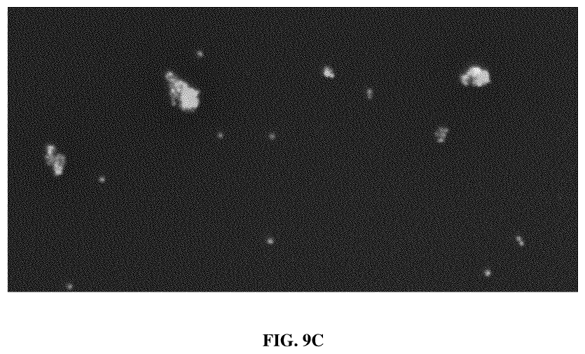

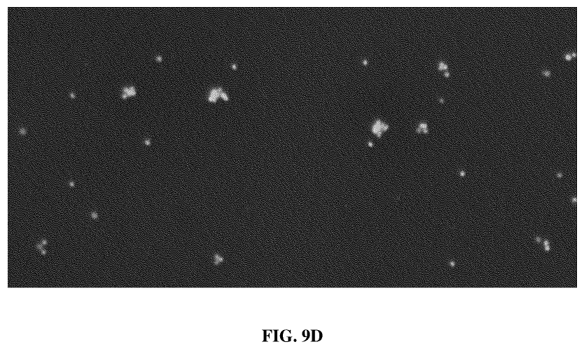

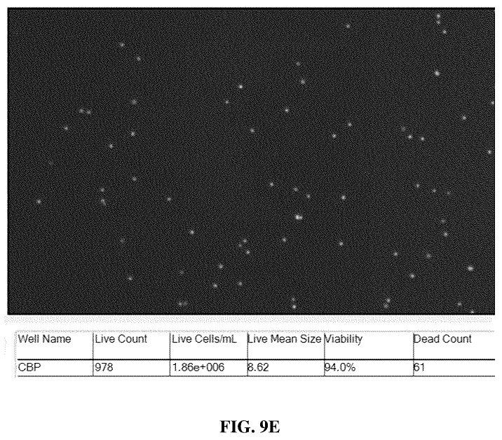

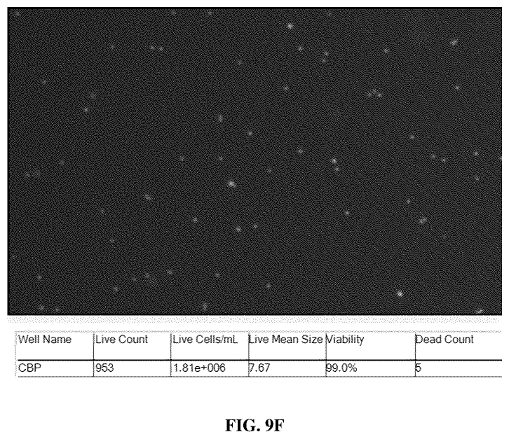

This analysis revealed two populations of dead cells present in the freshly thawed PRP aggregates-free floating dead cells, and dead cells associated with aggregates. Within 15 minutes at room temperature the morphology of the free-floating dead cells was noticeably altered. There was a loss of membrane contrast in the brightfield images (FIG. 8D) and the PI staining became diffuse (FIG. 8C) minutes, a large majority of the free floating dead cells were eliminated. The remaining dead cells were clustered around, or integrated within, aggregates (FIGS. 8F-8G). Incubation in 10×TRYPLE™ at 37° C. greatly accelerated the disappearance of dead cells. At 2 minutes, most of the free-floating dead cells were already gone. At 10 or 30 minutes at 37° C. there were even smaller numbers of free-floating dead cells (FIG. 9A-9D), however they were not completely eliminated. When the 30-minute sample was triturated, mixed with AOPI, and analyzed immediately, 6% of cells were categorized as dead cells (FIG. 9E). Interestingly, when the sample was incubated in AOPI for 15 minutes prior to analysis, only 1% dead cells remained (FIG. 9F). There was rapid elimination of the free floating dead cells using 10×TRYPLE™ at 37° C.

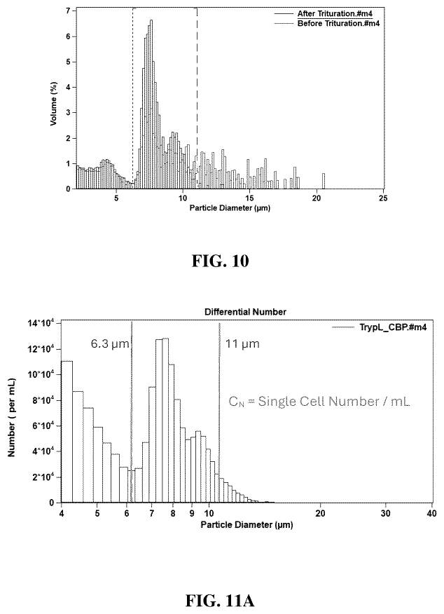

Coincident with these studies, evidence emerged that the osmolarity of the AOPI reagent (supplied by the commercial vendor for the CELLACA™) was lower than the osmolarity of the PRP carrier solution (BSS) or the basal medium used to formulate the quench reagent. The CELLACA™ assay requires a 2-fold dilution of cell samples in AOPI. Thus, it was speculated that the morphology of the PI labeled cells and the disappearance of those cell after incubation in AOPI (FIG. 10F) might be due to low osmolarity. Furthermore, it was considered whether this activity could be leveraged to bring the CELLACA™ and the Multisizer live cell counts into better alignment.

The small number of dead cells after a 30-minute incubation in 10×TRYPLE™ at 37° C. seemed to contradict the data showing 6-10% PI labeled cells in completely dissociated samples. However, it was reasoned that, for the complete dissociation method, the trituration and AOPI labeling steps caused sufficient damage to the membranes of live cells to allow penetration of the PI dye. This would explain the persistence of dead cells after trituration and AOPI labeling. This may also suggest that the complete dissociation method for complete dissociation may be too harsh to accurately estimate the concentration of live cells in the aggregate samples.

If the definition of a dead cell is specified as one that survives a 30-minute incubation in 10×TRYPLE™ at 37° C., then Multisizer counts from the completely dissociated samples would be the more accurate representation of the live cell concentration, despite the presence of membrane damaged cells. Similarly, this reasoning would suggest the percentage of viable cells is 10% higher than the calculated values obtained from the CELLACA™ data. However, it cannot be ruled out the possibility that the primary population of dead cells present at thaw remains present even after the TRYPLE™ incubation and the AOPI labeling. The results in FIG. 9 suggest dead cells present in PRP aggregates may be eliminated by incubation for 30-minutes in 10×TRYPLE™, and a subsequent incubation in a low osmolarity solution containing DNAase.

The PRP aggregates are difficult to dissociate. The TRYPLE™ enzyme incubation eliminates dead cells but it is insufficient to achieve complete dissociation. Automated cell counting algorithms are unable to de-cluster objects such as those shown in Figure #. Thus, the desire to use instruments such as the CELLACA™ or the ViCell requires employing mechanical shear forces, such as trituration achieve complete dissociation. However, if such sample preparation damages the cells, the result may be inaccurate reporting of cell concentrations, and inaccurate dose formulation.

Thus, it was sought to develop an orthogonal approach to cell counting that alleviated the need to apply trituration. Specifically, it was considered to use the data output from the Multisizer to convert biomass measurement of incompletely dissociated aggregate into meaningful live cell counts. To study the effect of trituration on the biomass distributions, a PRP sample was analyzed after the 30 minute incubation in TRYPLE™, and then reanalyzed the same sample after trituration, using the Multisizer (FIG. 10).

The TRYPLE™ treatment generates cell fragments and debris, as evidenced by the appearance of small particles less than 6 microns in size in both samples. Particles in a size range of 11 to 25 microns (clusters) were present prior to trituration. This is consistent with the imaging data in FIG. 8. Following the trituration step, very little biomass was present in particles larger than 11 microns and cells were completely dissociated into two single cell peaks.

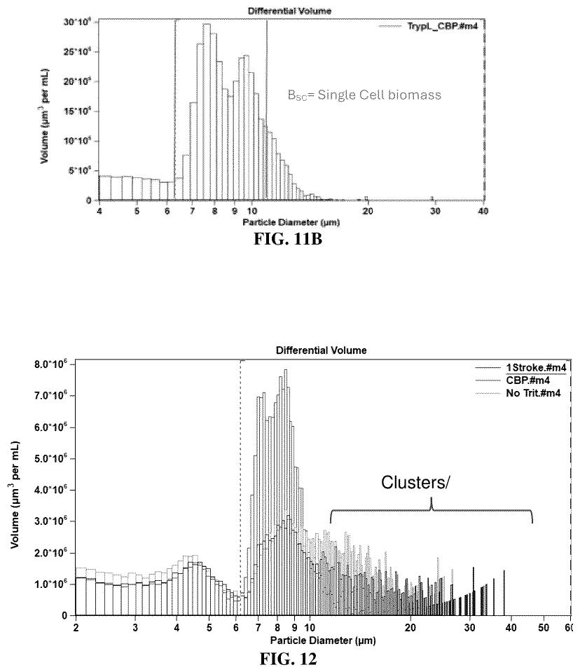

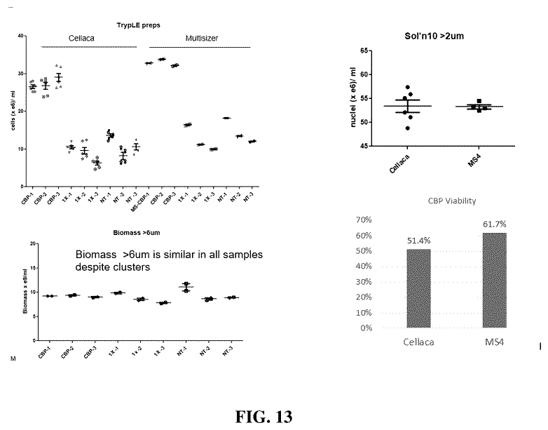

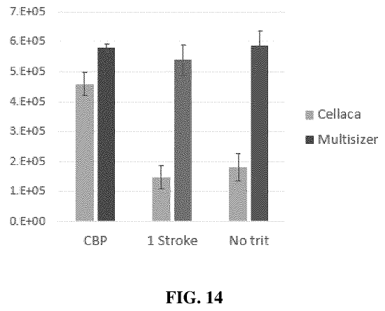

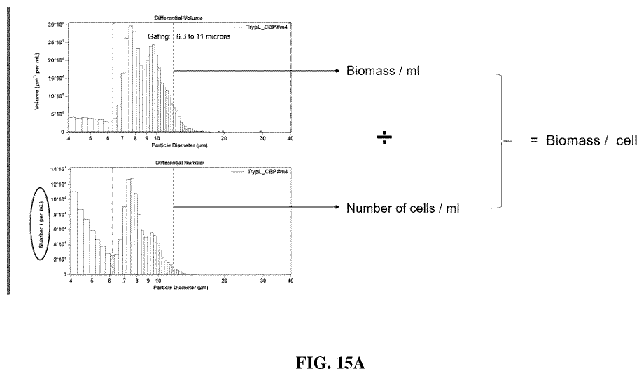

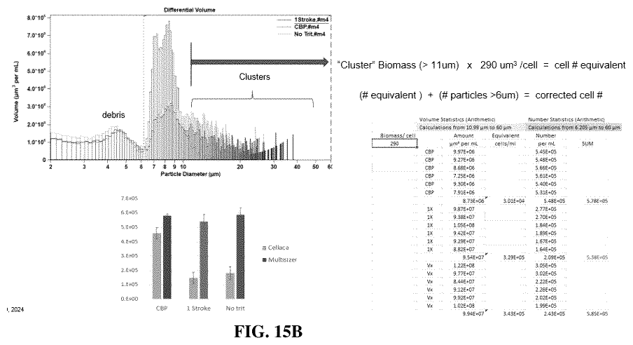

Recalling that the TRYPLE™ incubation eliminates dead cells, it was reasoned that biomass measurements might be useful in quantifying the average biomass per single live cell from a completely dissociated sample. This analysis involved 1) gating the particle counts histogram between 6.3 and 11 um to derive the concentration of single cells per ml (C_{N}), 2) then switching the Y-axis parameter to volume (or biomass) per ml and deriving the biomass per ml (C_{B}) within the same 6.3-11 um gate (FIG. 11). The latter step assumes that cells are spheres, and simply converts diameter measurements to biomass values using the equation for the volume of a sphere (Equation #1):

$$Volume of sphere , V = ( 4 / 3 ) π {r}^{3} .$$

With both values, C_{N} and C_{B}, the following equation may be solved to define the average biomass per single cell B_{SC} (Equation #2):

$$Biomass per single cell , {B}_{SC \cdot } = {C}_{B} \div {C}_{N}$$

The value of (B_{SC}.) biomass per cell may be defined for a processed sample provided that single cells are present in sufficient quantities so that it is obvious where to establish the gating parameters. Alternatively, a fixed value of B_{SC}. might be applied for each manufactured PRP lot.

Having a method to calculate B_{SC}., it was then asked whether this value could be used to calculate live cell numbers per ml from an incompletely dissociated sample. An experiment was designed to create three sets of 10×TRYPLE™ treated samples. Each sample was treated with TRYPLE™ for 30 minutes. A control sample was triturated 20 times (CBP), while a second and third set of samples received one stroke of trituration or no trituration. The samples were split and analyzed on the CELLACA™ or Multisizer.

Both particle counts/ml and biomass/ml values were obtained for gating parameters (6.3 to 11 microns) that define single cells measurements. The Multisizer analysis revealed incomplete dissociation in the test samples and a particle distribution profile that included single cells, clusters, and aggregates. As expected, these test samples produced significantly fewer live cell counts on the CELLACA™, and fewer Multisizer counts, compared to the completely dissociated samples. The Multisizer data was converted into biomass values (um³/ml), and a gating threshold >6.3 um was established to quantify the total biomass present in all live cells (B_{T}), including single cells, clusters, and aggregates. This analysis revealed very similar amounts of biomass in all samples.

A value for Biomass per single cell (B_{SC}.) was calculated and used to estimate cell concentration in the incompletely dissociated test samples, using the equation below Equation #3:

$$Cell concentration , {C}_{N} = {B}_{T} \div {B}_{SC \cdot }$$

The Multisizer is a particle counter that analyzes 12 different graphical parameters, including biomass concentration (volume/mL) and particulate count (number/mL) for each sample. Since the Multisizer cannot differentiate the individual cells found within a cluster, the particulate count (number/mL) of a partially dissociated sample will not be an accurate depiction of cell concentration compared a fully dissociated sample analyzed on the CELLACA™MX. However, it is proposed that utilizing the biomass parameter along with proper gating strategies and simple calculation can allow for accurate counts of a partially dissociated sample, permitting less trituration and therefore, less damage to live cell membranes.

After running a partially dissociated sample on the Multisizer, the biomass concentration (volume/mL) and particle count (number/mL) was gated from 6.3 microns to 11 microns as discussed previously as the range indicating only single cell diameter (FIG. 8A, 8B). These values were incorporated into Equation 1 to yield the biomass/cell value which is the amount of biomass corresponding to each single cell in the sample. This value fluctuates between different lots. Then, biomass/cell can be used to convert the biomass concentration of the entire sample (volume/mL) gated above 6.3 microns (FIG. 8C) to the total cell concentration of the sample (Equation 2).

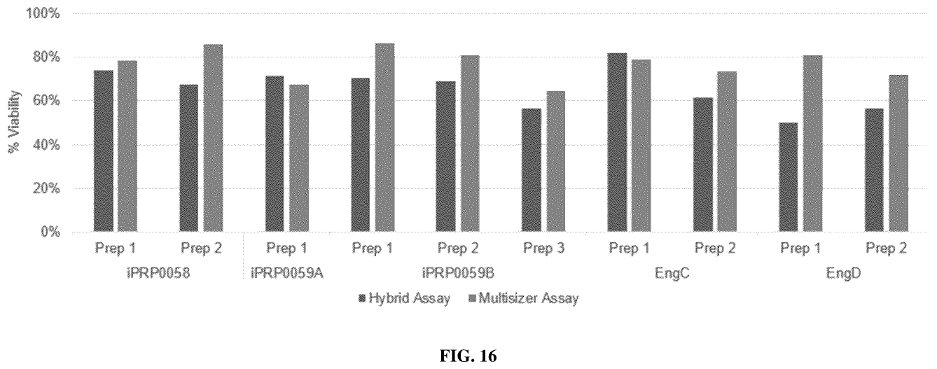

Percent viability was calculated using the cell counter method or the present particle counter (e.g., Multisizer). By using 10×TRYPLE™ to remove a significant portion of dead cells, viability was estimated (FIG. 16). The method of using the particle counter to determine percent viability by biomass was compared to the percent viability to the assay using cell counter with cell labeling.

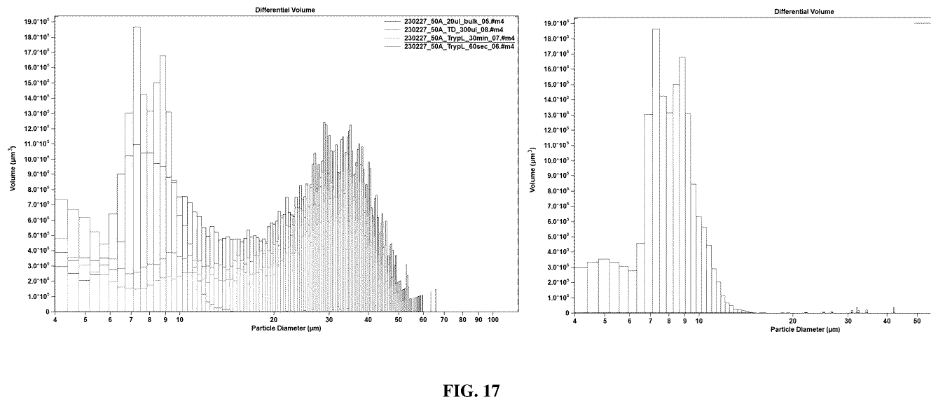

Further, differential volume of particles of different diameters was determined in a PRP cell aggregate sample using the Multisizer (FIG. 17). The red bars represent the 10×TRYPLE™ dissociated (TD) sample, after quench and trituration. The TD sample is not 100% fully dissociated as shown by some aggregates between 20 and 40 μm. The present methods of determining biomass (e.g., >6 microns) would capture these aggregates while a cell labeling method (e.g., using CELLACA™ or Nucleocounter) would not be able to resolve how many cells are in the aggregates. The blue bars are the bulk non-dissociated aggregates, 20 μl dispensed directly into a Multisizer cup with a defined volume of Isoton. This lot has poor percent aggregate biomass, as evidenced by the blue peak around 6-10 microns which are mostly individualized dead cells. After the bulk prep analysis, 1×TRYPLE™ was added directly to the same Multisizer cup and reanalyzed within 60 seconds as shown by the purple bars. This immediately knocked down the dead cell peak. The addition of TRYPLE™ here effectively diluted the sample by 10%, so the concentration of aggregates, and thus the magnitude of the 33 μm aggregate peak was reduced. However, interestingly, the mean size of the aggregates was unchanged. There are at least two explanations here: 1) there were no dead cells in the 50A aggregates, or 2) the removal of dead cells did not change the overall diameter of the aggregates. A closer look at the Aggregate-gated volume stats (>17 microns) suggest the latter explanation. The loss in biomass after 60 seconds was 23%, not 10%. Finally, the same sample was incubated at room temperature with stirring in 1×TRYPLE™ for 30 minutes (green bars). In the final analysis, the aggregates remained the same size, but the amount of biomass was further reduced by an additional 30%. The amount of aggregate biomass remaining was 34.93/64.55=54%.

The results here suggest an application for establishing the cell concentration of an aggregate cell sample without the need to completely dissociate the sample. This application would relieve the user of triturating the sample and eliminate the need to use fluorescent labeling dye for image analysis. This approach may apply broadly to any sample of clusters or aggregates of cells where by value of biomass per cell might be derived.

Thus, the present particle counter method to determine aggregate biomass can be used to reduce run to run and operator to operator variability without cell labeling. The bulk of the dead cells can be removed from an aggregate quickly and without a need to fully dissociate the cultures. While for the dye-based approach, a single cell state is needed to enable the visual resolution and segmentation of the live cell component, risking some level of cell loss and ultimately an underestimate of live cell content and perhaps increased variability, it would seem that the present particle counter assay does not require full dissociation of the samples to single cells.

### Example 2—CellTox Green Kit for Cell Viability Testing

Studies were performed on the use of the CellTox Green kit as an assay for use in viability testing for PRP aggregates. The CellTox Green kit was sourced from Promega and is designed to measures changes in membrane integrity that result from cell death. The dye in this assay binds to DNA and produces a fluorescent signal, which is detected with a plate reader.

A method was developed for determining viability, such as for PRP aggregates, involving generating two aliquots of the sample of interest, where one was labeled “lysed aggregates” and one was labeled “non-lysed aggregates”. Lysis buffer was added to the sample labeled “lysed aggregates” and triturated using the Gilson automated pipette 30 times. Both “lysed aggregates” and “non-lysed aggregates” were plated on black-walled plates. In addition to the samples, a DNA standard curve was plated. All samples were stained with CellTox Green dye, shaken briefly on a plate shaker, incubated shielded from light for 15 min, and read on a plate reader. CellTox Green is a DNA binding dye and therefore any cell with a compromised membrane will allow the dye to enter the cell and produce a fluorescent signal. Viability was calculated using the fluorescent readouts from the instrument as follows:

$$100 - ( \frac{“ non - lysed ”}{“ lysed ”} * 100 ) .$$

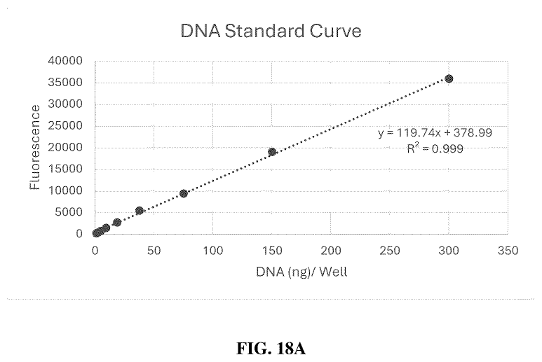

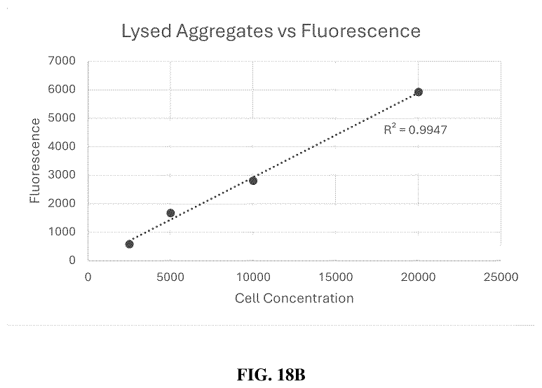

A DNA standard curve was performed with each assay to determine the fluorescence range of the assay (FIG. 18A). Samples were excluded from analysis if their fluorescence values did not fall within the DNA standard curve. The linear range of the assay was between 2,500 and 20,000 cells per well for the lysed sample (FIG. 18B).

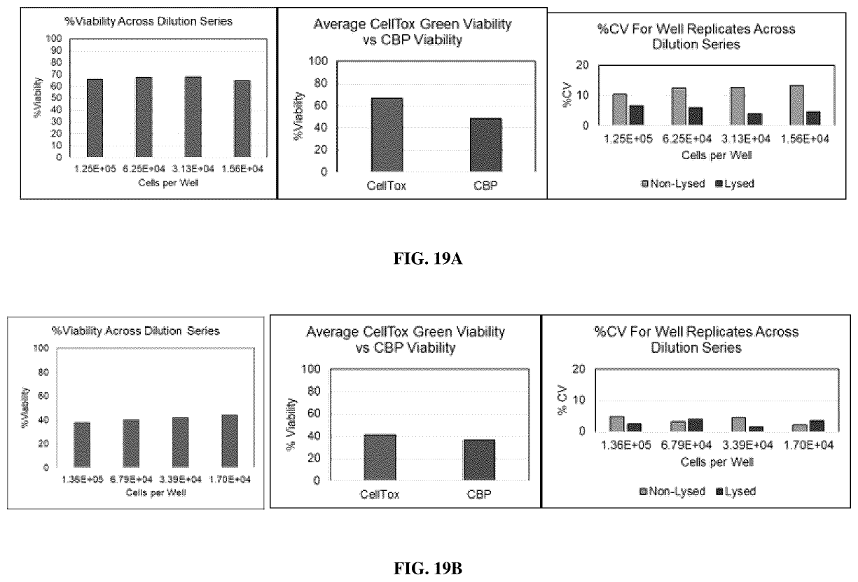

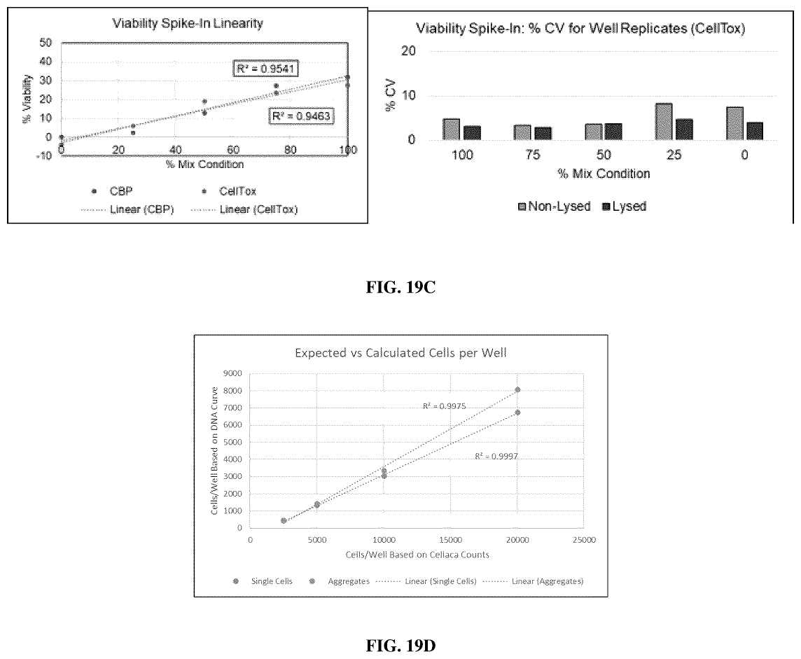

Across a series of cellular concentrations per well from 1.70e4 to 1.36e5, and measuring viability using the above method (CellTox Green), an approximately similar value was reported for viability at close to 40% (FIG. 19A). This was slightly higher than the value recorded using the complete dissociation method (current best practice (CBP)) assay using the CELLACA™, and the percent CV for fluorescent values across well replicates was <10%. This assay was performed on PRP aggregates stored in a refrigerator (4° C.) overnight in balanced salt solution (BSS). The assay was repeated using freshly thawed PRP aggregates. Similar consistency in viability was observed for a range of cell concentration per well, with percent CV under 20% for well-to-well replicates. Viability was higher using the CellTox assay, possibly due to using freshly thawed PRP aggregates (FIG. 19B). To test assay proportionality across a viability range, cells were killed by storing PRP aggregates in BSS overnight at 37 C, and these were mixed in controlled ratios with cells stored overnight at 4 C. Cells at 4 C have some viable cells. Mixing cells at controlled ratios led to proportional viability measurements across the series (FIG. 19C). When testing PRP as aggregates as aggregates and single cells, the assay was proportional across a range of concentrations. A new linear range of this assay was obtained with a high concentration of 20,000 cells/well (FIG. 19D).

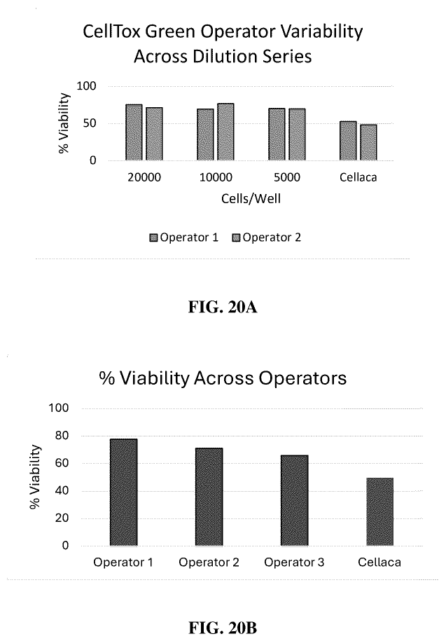

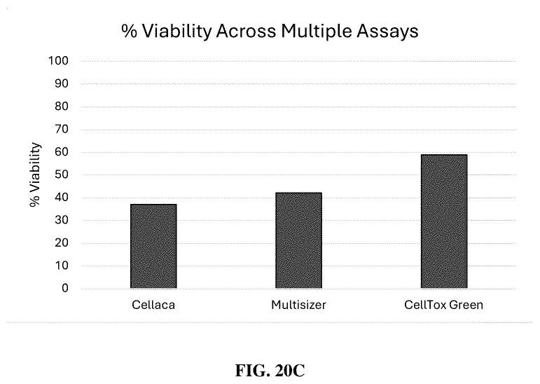

Percent viability was directly compared to the CELLACA™ using the new linear range, and viability was similar between operators and observed to be higher than viability recorded on the CELLACA™ (FIG. 20A). Viability across multiple operators was tested on the CellTox assay. All operators recorded higher viability than the CELLACA™, but there was some operator-to-operator variability (FIG. 20B). Using PRPs, the CellTox Green assay was directly compared to the CELLACA™ and Multisizer. The CellTox Green assay recorded higher percent viability than other viability assays (FIG. 20C).

All methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alexander et al., Proc. Nat. Acad. Sci. USA, 85:5092-5096,1988.
Barnea-Cramer et al. Sci Rep. 6:29784, 2016.
Bhutto and Lutty. Mol Aspects Med. 33(4):295-317, 2012.
Ercolani et al., J. Biol. Chem., 263:15335-15341,1988.
Hirami et al., Neurosci. Lett., 48: 126-131, 2009.
Hirami et al., Neurosci. Lett., 48: 126-131, 2009.
International Patent No. PCT/US2016/050543
International Patent No. PCT/US2016/050554
International Patent No. PCT/US2019/028557
International Publication No. WO 98/30679.
Karin et al. Cell, 36:371-379,1989.
Ludwig et al., Nat. Biotechnol., 24:185-187, 2006b.
Ludwig et al., Nat. Methods, 3:637-646, 2006a.
Macejak and Sarnow, Nature, 353:90-94, 1991.
Ng, Nuc. Acid Res., 17:601-615, 1989.
Oner. Turk J Ophthalmol. 48(1):33-8, 2018.
PCT Publication No. WO 2007/069666
PCT Publication No. WO 2014/121077.
Pelletier and Sonenberg, Nature, 334(6180):320-325, 1988.
Pennington and DeAngelis. Eye Vis (Lond). 3:34, 2016.
Richards et al., Cell, 37:263-272, 1984.
Sambrook and Russel, Molecular Cloning: A Laboratory Manual, 3rd Ed. Cold Spring Harbor Lab. Press, 2001.
Shimizu et al Cell Growth Differ 8: 1349-1358, 1997.
Strauss et al., Physiological Reviews, 85:845-881, 2005.
Strauss. Physiol Rev. 85(3):845-81, 2005.
Takahashi et al., Cell, 126, 663-676, 2006.
Takahashi et al., Cell, 131, 861-872, 2007.
Thomson and Marshall, Curr. Top. Dev. Biol., 38:133-165, 1998.
Thomson and Odorico, Trends Biotechnol., 18(2):53-57, 2000.
Thomson et al. Proc. Natl. Acad. Scie. USA, 92:7844-7848, 1995.
U.S. Patent Application No. 2002/0055144.
U.S. Patent Application No. 2002/0076747.
U.S. Patent Application No. 2009/0148425.
U.S. Patent Application No. 2009/0246875.
U.S. Patent Application No. 2010/0210014.
U.S. Patent Application No. 2012/0196360.
U.S. Patent Application No. 2012/0276636.
U.S. Pat. No. 4,683,202.
U.S. Pat. No. 5,556,954.
U.S. Pat. No. 5,843,780.
U.S. Pat. No. 5,925,565.
U.S. Pat. No. 5,928,906.
U.S. Pat. No. 5,935,819.
U.S. Pat. No. 6,103,470.
U.S. Pat. No. 6,200,806.
U.S. Pat. No. 6,416,998.
U.S. Pat. No. 6,833,269.
U.S. Pat. No. 7,029,913.
U.S. Pat. No. 7,442,548.
U.S. Pat. No. 7,598,364.
U.S. Pat. No. 7,682,828.
U.S. Pat. No. 7,989,425.
U.S. Pat. No. 8,058,065.
U.S. Pat. No. 8,071,369.
U.S. Pat. No. 8,129,187.
U.S. Pat. No. 8,268,620.
U.S. Pat. No. 8,546,140.
U.S. Pat. No. 8,741,648.
U.S. Patent Publication No. 2003/0211603.
U.S. Patent Publication No. 2010/0003757.
Wong et al. The Lancet Global Health. 2(2):e106-e1, 2014.
Yu et al., Science, 318:1917-1920, 2007.
Zhao et al. Development. 144(8):1368-81, 2017.
Zhou et al. Development. 142(19):3294-306, 2015.

1. A method of obtaining a live cell concentration of a cell aggregate composition comprising:
(a) obtaining a fraction of the cell aggregate composition;
(b) contacting the fraction of the cell aggregate composition with a dissociation enzyme to obtain a dissociated sample; and
(c) quantifying the number of live cells in the dissociated sample by counting the number of particles based on size to obtain a live cell concentration.

2. The method of claim 1, wherein the cell aggregate composition comprises cells derived from induced pluripotent stem cells (iPSCs).

3. The method of claim 2, wherein the cells derived from iPSCs are photoreceptor precursor cells (PRPs), photoreceptor cells, or retinal epithelial cells.

4. The method of claim 1, wherein the fraction is 1%-5% of the cell aggregate composition.

5. (canceled)

6. The method of claim 1, wherein the dissociation enzyme is TRYPLE™, ACCUTASE, trypsin, dispase, or papain.

7. The method of claim 1, further comprising treating dissociated sample with DNAse prior to step (c).

8. The method of claim 7, further comprising triturating the dissociated sample treated with a nuclease.

9. The method of claim 1, wherein quantifying the number of live cells comprises using a particle counting instrument configured for detection of single cells.

10. (canceled)

11. The method of claim 9, wherein the instrument configured for detection of single cells comprises counting particles with a diameter greater than 6.3 μm.

12. The method of claim 1, further comprising adjusting the live cell concentration to the total cell aggregate composition to obtain a target concentration for dose formulation.

13. The method of claim 1, wherein the cell aggregate composition comprises individual cells, cell clusters, and cell aggregates.

14. The method of claim 1, wherein the cell aggregate composition comprises cell clusters and/or cell aggregates.

15. The method of claim 1, wherein the method does not comprise using a labeling dye.

16. A method of obtaining a total cell concentration of a cell aggregate composition comprising:
(a) obtaining a fraction of the cell aggregate composition;
(b) contacting said fraction of the cell aggregate composition with a cell lysis solution to obtain a lysed sample; and
(c) quantifying the number of nuclei in the lysed sample by counting the number of particles based on size to obtain a total cell concentration.

17. The method of claim 16, wherein the fraction is 1%-5% of the cell aggregate composition.

18. (canceled)

19. The method of claim 1, wherein the cell lysis solution is Solution 10.

20. (canceled)

21. The method of claim 20, wherein the instrument configured for detection of nuclei comprises counting particles with a diameter greater than 2.8 μm.

22. The method of claim 1, wherein the cell aggregate composition comprises individual cells, cell clusters, and cell aggregates.

23. (canceled)

24. (canceled)

25. A method of determining the cell viability of a cell aggregate composition comprising:
(a) obtaining a live cell concentration by the method of claim 1; and
(b) obtaining a total cell concentration by the method of (i) obtaining a fraction of the cell aggregate composition;
(ii) contacting said fraction of the cell aggregate composition with a cell lysis solution to obtain a lysed sample; and
(iii) quantifying the number of nuclei in the lysed sample by counting the number of particles based on size to obtain a total cell concentration, wherein cell viability=live cell concentration/total cell concentration.

26. A method of determining the total live cell biomass of a cell aggregate composition comprising:
(a) contacting a cell aggregate composition with a dissociation enzyme to obtain a dissociated sample; and
(b) quantifying the number of single cells, cell clusters, and cell aggregates per mL in the dissociated sample by counting the number particles based on size, wherein the sum of biomass for each of the single cell population, cell cluster population, and cell aggregate population represents the total live cell biomass (LCB).

27.-35. (canceled)
